(19) United States
(12) Patent Application Publication
(10) Pub. No.: US 2003/0078223 A1
(43) Pub. Date: Apr. 24, 2003

(54) Compositions and methods for modulating an immune response

(76) Inventors: Eyal Raz, Del Mar, CA (US); David Broide, San Diego, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/099,512

(22) Filed: Mar. 15, 2002

Related U.S. Application Data
(63) Continuation-in-part of application No. 09/235,742, filed on Jan. 21, 1999.
(63) Continuation-in-part of application No. 08/927,120, filed on Sep. 5, 1997, now abandoned.
(63) Continuation-in-part of application No. 09/265,191, filed on Mar. 10, 1999.
(63) Continuation of application No. 08/593,554, filed on Jan. 30, 1996, now abandoned.
(60) Provisional application No. 60/276,865, filed on Mar. 16, 2001.

(51) Int. Cl. A61K 48/00; A61K 39/35

(52) U.S. Cl. 514/44; 424/275.1

(57) ABSTRACT

The present invention provides methods of maintaining suppression of a Th2 immune response, and methods of maintaining an increase in a Th1 immune response in an individual. The methods generally involve administering to an individual an effective amount of a first dose of a composition comprising an immunomodulatory nucleic acid, and, after a suitable time, administering at least a second dose of a composition comprising an immunomodulatory nucleic acid.

### CROSS-REFERENCE TO RELATED APPLICATIONS

This application claims the benefit of earlier-filed U.S. provisional application serial No. 60/276,865, filed Mar. 16, 2001; and is a continuation-in-part of U.S. patent application Ser. No. 09/235,742, filed Jan. 21, 1999, which is a continuation-in-part of U.S. patent application Ser. No. 08/927,120, filed Sep. 5, 1997; and is a continuation-in-part of U.S. patent application Ser. No. 09/265,191, filed Mar. 10, 1999, which is a continuation of U.S. patent application Ser. No. 08/593,554, filed Jan. 30, 1996, which applications are incorporated herein by reference in their entirety.

### STATEMENT OF GOVERNMENT RIGHTS

This invention was made with Government support under Grant No. AI37305, awarded by the National Institutes of Health. The Government may have certain rights in this invention.

### FIELD OF THE INVENTION

The invention relates to methods and oligonucleotide compositions for use modulating a host immune response, particularly in reducing or suppressing a host immune response associated with allergy.

### BACKGROUND OF THE INVENTION

Allergic asthma is characterized by cellular infiltration of the airways with eosinophils and T lymphocytes expressing a Th2 profile of cytokines. This characteristic inflammatory response is evident both in bronchial biopsies obtained from asthmatic patients as well as in mouse models of altered airway responsiveness. Following allergen inhalation in sensitized subjects or animals, Th2 cells release a particular set of cytokines (i.e., IL-5, GM-CSF and IL-3) that promote airway eosinophilia by several different mechanisms, including induction of eosinophil proliferation in the bone marrow, promotion of the release of cosinophils from the bone marrow, and inhibition of eosinophil apoptosis. In addition to promoting airway eosinophilia, these Th2 cytokines prime and activate eosinophils to release proinflammatory cytoplasmic granule products, lipid mediators, and cytokines that are thought to contribute to the tissue damage, remodeling, and hyperresponsiveness of the asthmatic airways.

Antiinflammatory medications such as corticosteroids are standard therapy for asthma, but have limitations in that they may not be disease modifying (asthma recurs when the corticosteroids are discontinued). In addition, corticosteroids, even when delivered by the inhalation route, are associated with the potential for significant side effects, including cataracts, growth retardation, and osteoporosis. Therefore, there is a need to develop safe and effective alternative therapies to corticosteroids to inhibit the critical events (e.g., Th2 cell activation) that initiate and perpetuate eosinophilic inflammation in the airways.

At present there is limited information regarding the duration of effect of a single dose of ISS and whether the effect of ISS in inducing a switch from a Th2 to a Th1 response is transient or permanent. These issues are important in understanding the dosing frequency of ISS as a therapeutic agent to maintain the inhibition of Th2 responses associated with allergic diseases such as asthma. In addition, it is important to know whether the ISS immunomodulating effect is permanent or transient as concern has been expressed about irreversibly inhibiting Th2 responses when such Th2 immune responses may potentially be needed in host immune defense. Thus, there is a need in the art for development of immunization protocols that optimize the timing of administration of ISS.

The present invention addresses this need by providing dosing regimens that take into account the duration of the effect of ISS on an immune response.

### SUMMARY OF THE INVENTION

The present invention provides methods of maintaining suppression of a Th2 immune response, and methods of maintaining an increase in a Th1 immune response in an individual. The methods generally involve administering to an individual an effective amount of a first dose of a composition comprising an immunomodulatory nucleic acid, and, after a suitable time, administering at least a second dose of a composition comprising an immunomodulatory nucleic acid.

### BRIEF DESCRIPTION OF DRAWINGS

FIG. 1 is a chart which summarizes aspects of the mammalian immune system.

FIG. 2 is a graph of data which confirm a shift from a T2 to a Th1 phenotype (as measured by IgG2A production) in mice treated with an ISS-ODN 3 days before antigen challenge.

FIG. 3A and 3B are graphs of data which confirm the induction of a Th2 phenotype (as measured by IgGI production) in mice treated with a mutant, inactive ISS-ODN 3 days before antigen challenge.

FIG. 4 is a graph of data which confirm Th1-associated suppression of antigen-specific IgE in antigen-sensitized, ISS (pCMV-LacZ, a plasmid containing two copies of an ISS) treated mice as compared to antigen-sensitized (control) mice.

FIG. 5 is a graph of data which confirm suppression of IL-4 secretion by ISS-ODN as compared to a control.

FIG. 6 is a graph of data which confirm suppression of IL-5 secretion by ISS-ODN as compared to a control.

FIG. 7 is a graph of data which confirm suppression of IL-10 secretion by ISS-ODN as compared to a control.

FIG. 8 is a graph of data which confirm stimulation of IFN-γ secretion by ISS-ODN as compared to a control.

FIG. 9 is a graph of data demonstrating an ISS-ODN mediated shift to a Th1 phenotype (as indicated by IFN-γ levels) in animals treated with ISS-ODN before antigen challenge (asterisked bars) in comparison to those co-treated with ISS-ODN and antigen.

FIG. 10 is a graph of data demonstrating an ISS-ODN mediated boost in immune responsiveness (as indicated by increases in CD4⁺ lymphocyte proliferation) in animals treated with ISS-ODN before antigen challenge (asterisked bars) in comparison to those co-treated with ISS-ODN and antigen.

FIGS. 11a and 11b are graphs showing production of anti-NP antibodies following injection with a plasmid encoding viral NP.

FIG. 12 is a schematic representation of pCMV-LacZ.

FIGS. 13a, 13b, and 13c are plasmid maps of pKCB-Z, pKCB 1aa-z, and pKCB 2aa-z, respectively.

FIG. 14 is a graph depicting production of antibodies to β-galactosidase in animals injected with various plasmids.

FIG. 15 is a graph depicting β-galactosidase activity in Chinese hamster ovary cells transfected separately various vectors.

FIG. 16 is a graph depicting production of antibodies to β-galactosidase in animals injected with various plasmids.

FIG. 17 is a graph depicting production of antibodies to β-galactosidase in animals injected with various plasmids.

FIG. 18 is a graph depicting CTL activity in cultures of cells from the mice injected with various plasmids.

FIG. 19 is a graph depicting CTL activity in cultures of cells from the mice injected with various plasmids.

FIG. 20 is a graph depicting the effect of intradermal gene immunization on viral challenge.

FIG. 21 is a graph depicting the immune response to β-galactosidase in mice injected i.d. with pCMV-lacZ, injected i.m. with pCMV-LacZ, or injected i.m. with β-galactosidase.

FIG. 22 is a graph depicting the production of IgG2a antibodies to β-galactosidase.

FIG. 23 is a graph depicting the production of IgGI antibodies to β-galactosidase.

FIG. 24 is a graph depicting production of IgG2a antibodies to β-galactosidase.

FIG. 25 is a graph depicting the production of IgGI antibodies to β-galactosidase.

FIG. 26 is a graph depicting the production of IgE antibodies to β-galactosidase.

FIG. 27 is a graph depicting the results of an ELISA to measure serum levels of anti-NP IgG following epidermal administration of the pCMV-NP vector with the application of a chemical agent.

FIG. 28 is a schematic representation of a protocol for ISS administration and antigen sensitization and challenge.

FIG. 29 is a table depicting the effect of ISS on IL-5 and IFN-γ levels.

FIG. 30 is a table depicting the effect of ISS on BAL and lung eosinophils.

FIG. 31 is a table depicting the effect of ISS on methacholine PC200 airway responsiveness.

FIG. 32 is a table depicting the effects of ISS on IgE levels.

### DETAILED DESCRIPTION OF THE INVENTION

The present invention provides methods of maintaining suppression of a Th2 immune response, and methods of maintaining an increase in a Th1 immune response in an individual. The methods generally involve administering to an individual an effective amount of a first dose of a composition comprising an immunomodulatory nucleic acid, and, after a suitable time, administering at least a second dose of the composition.

The invention provides means to prevent onset of, or rapidly suppress, antigen-stimulated inflammation in a host by administration of an immunomodulatory nucleic acid molecule, commonly referred to as an immunostimulatory nucleic acid molecule (ISS). The immunomodulatory nucleic acid may be administered without the need for co-administration of an antigen to which the host is sensitized, although such is optional and in some embodiments may be desired. Specifically, the invention provides methods for protecting an antigen-sensitized mammalian host against subsequent antigen challenge. Where the method of the invention involves administration of immunomodulatory nucleic acid without antigen, the invention provides therapy for allergy without the risk of anaphylaxis. In addition, the methods of the invention provide for suppression of one or more symptoms of antigen-induced inflammation for several days to weeks after administration of immunomodulatory nucleic acid.

Immunomodulatory nucleic acids have anti-inflammatory properties in addition to their immunostimulatory properties. Such nucleic acids are therefore useful in the treatment and prevention of inflammation associated with allergy, including, but not necessarily limited to, antigen-stimulated granulocyte infiltration of tissue, such as occurs in the respiratory passages of asthmatics during an asthma attack. Advantageously, delivery of immunomodulatory nucleic acid according to the invention suppresses antigen-stimulated granulocyte infiltration into host tissue even before the immunomodulatory nucleic acid affects the host's immune response to the antigen. Thus, the invention provides an antigen-independent method to reduce antigen-stimulated inflammation by suppressing cellular adhesion, thereby avoiding the release of inflammatory mediators which would be stimulated through granulocyte-binding of endothelial cells.

An example of a therapeutic application for the invention is in the control of asthma, whereby the immunomodulatory nucleic acid is delivered into pulmonary tissue intranasally or by systemic routes. In asthmatics, eosinophil infiltration of lung tissue occurs mainly during the late phase of an allergic response to a respiratory allergen. Canonical immunotherapy can modulate the host immune response to the allergen and eventually stem the tide of eosinophils into the host airways. However, practice of the invention suppresses eosinophil infiltration of host airways well before the host immune system responds to the respiratory allergen, thereby providing a form of protection against the airway narrowing and respiratory tissue damage, which characterize an acute asthma attack.

In another aspect, the invention provides means to shift a present host cellular immune response to an antigen away from a Th2 phenotype and into a Th1 phenotype. To this end, immunomodulatory nucleic acids are delivered by any route through which antigen-sensitized host tissues will be contacted with the immunomodulatory nucleic acid. Unlike canonical immunotherapy, immunity is stimulated by this method of the invention even when no additional antigen is introduced into the host. Thus, use of the method to boost the immune responsiveness of a host to subsequent challenge by a sensitizing antigen without immunization avoids the risk of immunization-induced anaphylaxis, suppresses IgE production in response to the antigen challenge and eliminates the need to identify the sensitizing antigen for use in immunization. An especially advantageous use for this aspect of the invention is treatment of localized allergic responses in target tissues where the allergens enter the body, such as the skin and mucosa.

Suppression of the Th2 phenotype according to the invention is also a useful adjunct to canonical immunotherapy to reduce antigen-stimulated IL-4 and IL-5 production. Thus, the invention encompasses delivery of immunomodulatory nucleic acid to a host to suppress the Th2 phenotype associated with conventional antigen immunization (e.g., for vaccination or allergy immunotherapy).

The shift to a Th1 phenotype achieved according to the invention is accompanied by increased secretion of IFN α, β, and γ, as well as IL-12 and IL-18.

Pharmaceutically acceptable compositions of ISS-ODN are provided for use in practicing the methods of the invention. The immunomodulatory nucleic acid of the invention include DNA or RNA oligonucleotides which are enriched with CpG dinucleotides, including those which are comprised of the primary structure 5′-Purine-Purine-C-G-Pyrimidine-Pyrimidine-3′.

Where appropriate to the contemplated course of therapy, the immunomodulatory nucleic acid may be administered with other anti-inflammatory or immunotherapeutic agents. Thus, a particularly useful composition for use in practicing the method of the invention is one in which an anti-inflammatory agent (e.g., a glucocorticoid) or immunotherapeutic agent (e.g., an antigen, cytokine or adjuvant) is mixed with an immunomodulatory nucleic acid.

The immunomodulatory nucleic acid can also be provided in the form of a kit comprising immunomodulatory nucleic acid and any additional medicaments, as well as a device for delivery of the immunomodulatory nucleic acid to a host tissue and reagents for determining the biological effect of the immunomodulatory nucleic acid on the treated host.

Definitions

The terms “immunomodulatory nucleic acid molecule,” “immunomodulatory nucleic acid,” “immunostimulatory nucleic acid molecule,” “ISS,” “ISS-PN,” and “ISS-ODN,” are used interchangeably herein, and without limitation, and refer to a polynucleotide that comprises at least one immunomodulatory nucleic acid moiety. The term “immunomodulatory,” as used herein in reference to a nucleic acid molecule, refers to the ability of a nucleic acid molecule to modulate an immune response in a vertebrate host, particularly a mammalian host.

The terms “oligonucleotide,” “polynucleotide,” and “nucleic acid molecule”, used interchangeably herein, refer to polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites, and/or phosphorothioates, and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. The polynucleotide may comprise one or more L-nucleosides. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be modified to comprise N3′-P5′ (NP) phosphoramidate, morpholino phosphorociamidate (MF), lockaed nucleic acid (LNA), 2′-O-methoxyethyl (MOE), or 2′-fluoro, arabino-nucleic acid (FANA), which can enhance the reistance of the polynucleotide to nuclease degradation (see, e.g., Faria et al. (2001) Nature Biotechnol. 19:40-44; Toulme (2001) Nature Biotechnol. 19:17-18). A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Immunomodulatory nucleic acid molecules can be provided in various formulations, e.g., in association with liposomes, microencapsulated, etc., as described in more detail herein.

The terms “polypeptide,” “peptide,” and “protein”, used interchangeably herein, refer to a polymeric form of amino acids of any length, which can include coded and non-coded amino acids, chemically or biochemically modified or derivatized amino acids, and polypeptides having modified peptide backbones. The term includes polypeptide chains modified or derivatized in any manner, including, but not limited to, glycosylation, formylation, cyclization, acetylation, phosphorylation, and the like. The term includes naturally-occurring peptides, synthetic peptides, and peptides comprising one or more amino acid analogs. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “antigen” is a term well understood in the art. Antigens can be of any class of macromolecule, including polypeptides, polysaccharides, lipopolysaccharides, glycoproteins, lipoproteins, and the like. As used herein, the term “antigen” includes antigenic polypeptides; antigenic fragments of polypeptides; epitopes; polyvalent conjugates of multiple epitopes linked to a solid support or a non-immunogenic macromolecule; and the like.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, preferably 75% free, and most preferably 90% free from other components with which it is naturally associated.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as particular lymphocyte populations. The term “biological sample” encompasses a clinical sample, and also includes cells in culture, cell supernatants, cell lysates, serum, plasma, biological fluid, and tissue samples.

“Treatment” or “treating” as used herein means any therapeutic intervention in a subject, usually a mammalian subject, generally a human subject, including: (i) prevention, that is, causing the clinical symptoms not to develop, e.g., preventing infection and/or preventing progression to a harmful state; (ii) inhibition, that is, arresting the development or further development of clinical symptoms, e.g., mitigating or completely inhibiting an active inflammatory process, which decrease can include complete elimination of inflammation in the subject (e.g., at the site treated or systemically); and/or (iii) relief, that is, causing the regression of clinical symptoms, e.g., causing a relief of inflammation or symptoms of inflammation (e.g., IgE levels, histamine, reduction in inflammatory cytokines (e.g., IL-5, and the like).

The term “effective amount” or “therapeutically effective amount” means a dosage sufficient to provide for treatment for the disease state being treated or to otherwise provide the desired effect (e.g., induction of an effective immune response). The precise dosage will vary according to a variety of factors such as subject-dependent variables (e.g., age, immune system health, etc.), the disease (e.g., the particular type or source of antigen-induced inflammation), and the treatment being effected.

By “subject” or “individual” or “patient” is meant any subject for whom or which therapy is desired. Suitable subjects include mammals. Human subjects are of particular interest. Other subjects may include non-human primates, cattle, sheep, goats, dogs, cats, birds (e.g., chickens or other poultry), guinea pigs, rabbits, rats, mice, horses, and so on. Of particular interest in many embodiments are subjects having or susceptible to antigen-induced inflammation.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an immunomodulatory nucleic acid” includes a plurality of such nucleic acids and reference to “the symptom” includes reference to one or more symptoms and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Overview

The present invention is based in part on the discovery that immunomodulatory nucleic acid, when administered to an antigen-sensitized host, can reduce or prevent inflammation (including symptoms of inflammation) upon subsequent exposure to the antigen to which the host is sensitized (e.g., antigen-induced inflammation in an immunomodulatory nucleic acid-treated, antigen-sensitized host is reduced relative to antigen-induced inflammation in an untreated, antigen-sensitized host).

The invention is further based on the discovery that the effects of immunomodulatory nucleic acid administration in reducing inflammation (including symptoms of antigen-induced inflammation) are long lasting. For example, symptoms of antigen-induced inflammation are reduced in the antigen-sensitized host for about 1-6 days, about 1 week, about 2 weeks, about 4 weeks, about 6 weeks, up to about 8 weeks following immunomodulatory nucleic acid administration. This discovery not only provides evidence that the effects of immunomodulatory nucleic acid are relatively long-lived, but also provides guidance for administration of booster doses of immunomodulatory nucleic acid, e.g., provides guidance for the timing of administration of immunomodulatory nucleic acid of subsequent doses to the host.

The present invention provides methods for modulating an immune response to an antigen. In some embodiments, the methods generally involve administering to an individual who is sensitized to an antigen an immunostimulatory nucleic acid molecule. In many embodiments, the immunostimulatory nucleic acid molecule does not encode the antigen to which the individual is sensitized. In these embodiments, the immunostimulatory nucleic acid molecule is generally administered without the antigen to which the individual is sensitized.

The present invention further provides methods for modulating an immune response to an antigen, generally involving administering to a naive individual an immunomodulatory nucleic acid. In some of these embodiments, an antigen is administered along with the immunomodulatory nucleic acid. In some embodiments, the antigen itself is administered. In other embodiments, the antigen is encoded by a polynucleotide. In some embodiments, the polynucleotide that encodes the antigen also includes the immunomodulatory nucleic acid. In other embodiments, the polynucleotide that encodes the antigen and the immunomodulatory nucleic acid are physically separate, e.g., are not physically linked.

In one aspect the invention provides a method for preventing or reducing antigen-stimulated, granulocyte-mediated inflammation in a tissue of an antigen-sensitized mammalian host comprising delivering an immunostimulatory oligonucleotide (ISS-ODN) to the host; wherein a reduction in, or the absence of, a Th2 type immune response measured in the host; or a reduction in, or the absence of other clinical signs of inflammation in the host after antigen challenge, indicates that the desired prevention or reduction in granulocyte-mediated inflammation has been achieved.

In another aspect, the invention provides a method for boosting the immune responsiveness of a mammalian host to a sensitizing antigen without immunization of the host by the sensitizing antigen comprising delivering an immunostimulatory oligonucleotide (ISS-ODN) to the host to the host, wherein an increase in the magnitude of the host immune response to the sensitizing antigen indicates that the desired boost to the host immune responsiveness has been achieved. In some embodiments, the host is suffering from asthma and the host's immune responsiveness to a respiratory allergen is boosted.

The methods are useful for treating a host, wherein the host is suffering from an inflammatory condition induced by the sensitizing antigen selected from the group of inflammatory conditions consisting of asthma, nasal polyposis, allergic rhinitis, atopic dermatitis, allergic conjunctivitis, eosinophilic fasciitis, ideopathic hypereosinophilic syndrome and cutaneous basophil hypersensitivity. In some embodiments, the inflamed tissue is skin or mucosa. In other embodiments, the inflamed tissue is respiratory tissue. In still other embodiments, the host is suffering from asthma.

In another aspect, the invention provides a method for shifting the immune response of a mammal host to a sensitizing antigen toward a Th1 phenotype comprising delivering an immunostimulatory oligonucleotide (ISS-ODN) to the host, wherein detection of a Th1 type immune response by the host indicates that the desired shift to the Th1 phenotype has been achieved. In some embodiments, the host is suffering from asthma and the shift to the Th1 phenotype reduces eosinophil infiltration of the host lung tissue. In some embodiments, the host is suffering from an intracellular infection by a pathogen and the shift to the Th1 phenotype strengthens the host immune response to the pathogen. In some embodiments, the pathogen is a virus. In some embodiments, the host is suffering from reduced blood flow to a tissue and the shift to the Th1 phenotype stimulates angiogenesis in the treated tissue. In some embodiments, the host is suffering from diabetic retinopathy.

In some embodiments, the immunomodulatory nucleic acid comprises 5′-CG-3′, and in other specific embodiments comprises a hexameric nucleotide sequence consisting of 5′-CG-3′, e.g., 5′-X₁X₂CGX₃X₄-3′, where each of X₁, X₂, X₃, and X₄ are nucleotides. In other embodiments, the immunomodulatory nucleic acid comprises a hexameric nucleotide sequence 5′-Purine-Purine-C-G-Pyrimidine-Pyrimidine-3′. In other embodiments, the hexameric nucleotide sequence consists of AACGTT. In other embodiments, the hexameric nucleotide sequence is selected from the group of sequences: AGCGTC, GACGTT, GGCGTT, AACGTC, AGCGTC, GACGTC, GGCGTC, AACGCC, AGCGCC, GACGCC, GGCGCC, AGCGCT, GACGCT, GGCGCT, TTCGAA, GGCGTT and AACGCC. In other embodiments, the ISS-ODN is conjugated to an immunostimulatory or anti-inflammatory partner selected from the group consisting of non-antigenic polypeptides, antigenic polypeptides, polysaccharides, antibodies, glycoproteins, lipids and steroids.

In some embodiments, the effect on the immune response is measured by determining any of the following values in a sample containing lymphocytes obtained from the ISS-ODN treated host:

(1) a reduction in levels of IL-4, IL-5 and/or IL-10 measured before and after antigen challenge or detection of lower levels of IL-4, IL-5 and/or IL-10 in the IS S-ODN treated host as compared to an antigen-challenged control;

(2) an increase in levels of IL-12, IL-18 and/or IFNα, IFNβ, or before and after antigen challenge or detection of higher levels of IL-12, IL-18 and/or interferon-alpha (IFN-α), interferon-beta (IFN-β), and interferon-gamma (IFN-γ) in the ISS ODN treated host as compared to an antigen-challenged control;

(3) IgG2a antibody production in the ISS-ODN treated host; or

(4) a reduction in levels of antigen-specific IgE as measured before and after antigen challenge or detection of lower levels of antigen-specific IgE in the ISS-ODN treated host as compared to an antigen-challenged control.

In some embodiments, reduction or suppression of inflammation is measured by assaying inflammatory infiltrate from the host for a reduction in granulocyte counts in inflammatory infiltrate of an affected host tissue as measured in an antigen challenged host before and after ISS-ODN administration or detection of lower levels of granulocyte counts in an ISS-ODN treated host as compared to an antigen-challenged control.

In another aspect, the invention provides a kit for use in reducing or preventing inflammation in an antigen-sensitized host tissue, as well as in boosting the immune responsiveness of a host to a sensitizing antigen, comprising an immunostimulatory oligonucleotide (ISS-ODN) in a sterile vial, a device for delivering the ISS-ODN directly into a host tissue and at least one assay reagent for use in measuring any of the following values as indicators that the desired reduction or prevention of inflammation or boost in immune responsiveness has been achieved in an ISS-ODN treated host:

(1) a reduction in levels of IL-4, IL-5 and/or IL-10 measured before and after antigen challenge; or detection of lower (or absent) levels of IL-4, IL-5 and/or IL-10 in an ISS-ODN treated host as compared to an antigen-primed, or primed and challenged, control;

(2) an increase in levels of IL-12, IL-18 and/or interferon-alpha (IFN-α), interferon-beta (IFN-β), and interferon-gamma (IFN-γ) before and after antigen challenge; or detection of higher levels of IL-12, IL-18 and/or interferon-alpha (IFN-α), interferon-beta (IFN-β), and interferon-gamma (IFN-γ) in an ISS ODN treated host as compared to an antigen-primed, or primed and challenged, control;

(3) IgG2a antibody production in an ISS-ODN treated host; or

(4) a reduction in levels of antigen-specific IgE as measured before and after antigen challenge; or detection of lower (or absent) levels of antigen-specific IgE in an ISS-ODN treated host as compared to an antigen-primed, or primed and challenged, control.

In another aspect, the invention provides a method for delivery of immunomodulatory nucleic acid to an antigen-sensitized host in a manner that provides for prolonged protection from antigen-induced inflammation that can normally be caused in the host due to antigen exposure.

In general, “booster” doses of immunomodulatory nucleic acid can be administered at about 1 day, 2 day, 3 day, 4 day, 5 day, about 1 week, about 2 week, about 4 week, about 6 week, or about 8 week intervals, in order to provide for reduction of one or more symptoms of antigen-induced inflammation (e.g., reduction of inflammatory cytokine levels, reduction in IgE levels, and the like). In general, one or more symptoms of antigen-induced inflammation are reduced at least about 30%, at least about 40-50%, at least about 70-75%, at least about 80% up to about 90-95% or more relative to symptoms that would normally be induced in the host upon antigen exposure in the absence of administering an immunomodulatory nucleic acid molecule, as described herein.

In another aspect, the invention provides a kit for use in reducing or preventing inflammation in an antigen-sensitized host tissue comprising an immunostimulatory oligonucleotide (ISS-ODN) in a sterile vial, a device for delivering the ISS-ODN directly into a host tissue and at least one assay reagent for use in measuring lymphocyte proliferation, IgG2a antibody levels, serum cytokine levels and/or granulocyte counts in inflammatory infiltrate of an affected host tissue.

Anti-Inflammatory and Immunotherapeutic Methods of the Invention

The present invention provides methods of maintaining suppression of a Th2 immune response in an individual, and methods of maintaining stimulation of a Th1 immune response in an individual. The methods generally involve administering to the individual a first dose of a composition comprising an immunomodulatory nucleic acid; and administering to the host at least a second dose of a composition comprising an immumodulatory nucleic acid, wherein the second dose is administered from about 1 day to about 8 weeks after the first dose.

The interval between administration of the first dose and the second dose is about 1 day, about 2 days, about 3 days, about 4 days, about 5 days, about 6 days, about 1 week, about 2 weeks, about 3 weeks, about 4 weeks, about 5 weeks, about 6 weeks or about 7 weeks. In many embodiments, further doses are administered at intervals which may be the same as or different from the interval between the first and second dose. Generally, intervals between any two subsequent consecutive doses is about 1 day, about 2 days, about 3 days, about 4 days, about 5 days, about 6 days, about 1 week, about 2 weeks, about 3 weeks, about 4 weeks, about 5 weeks, about 6 weeks or about 7 weeks.

The first and second doses, as well as subsequent doses, may be delivered by the same or different routes of administration. Furthermore, the amount of immunomodulatory nucleic acid in the first and second doses, as well as subsequent doses, may be varied, with the proviso that amount of immunomodulatory nucleic acid (and optionally, antigen) present in each dose is sufficient maintain a desired immune response profile (e.g., elevated Th1 and reduced Th2 relative to such responses in the absence of immunomodulatory nucleic acid therapy).

In some embodiments, the individual is sensitized to an antigen. In other embodiments, the individual is naive, e.g., the individual has not been administered a selected antigen, and/or has not been accidentally or otherwise exposed to the antigen.

In some embodiments, the immunomodulatory nucleic acid is administered without co-administration of an antigen, e.g., the immunomodulatory nucleic acid is administered without antigen per se, or without a polynucleotide encoding the antigen (e.g., where the antigen is a protein), e.g., where the polynucleotide does not comprise a coding sequence for the antigen that is capable of being expressed in a eukaryotic cell. In these embodiments, the immunomodulatory nucleic acid typically does not include a nucleotide sequence that encodes the antigen. In many of these embodiments, the individual is sensitized to an antigen (e.g., an allergen), and the antigen is not administered to the individual; instead, the individual is exposed to the antigen accidentally, inadvertently, or otherwise unintentionally, e.g., via environmental exposure, exposure via inhalation of antigen-containing air, ingestion of antigen-containing food, and the like.

In other embodiments, the first dose of immunomodulatory nucleic acid is co-administered with an antigen. In other embodiments, the second dose of immunomodulatory nucleic acid is co-administered with an antigen. In other embodiments, the first and the second dose of immunomodulatory nucleic acid are co-administered with antigen. In some embodiments, antigen is co-administered with a subsequent dose.

In some embodiments, antigen is co-administered with one or more doses of immunomodulatory nucleic acid, and the antigen is one to which the individual is sensitized. In these embodiments, the antigen is generally administered in very low amounts, as is well known in the art, to desensitize the individual to the antigen.

In some embodiments, antigen is co-administered with one or more doses of immunomodulatory nucleic acid, and the antigen is one to which the individual is not sensitized; instead, the antigen is one to which a Th1 immune response is stimulated using a subject method. Non-limiting examples of such antigens include viral antigens, bacterial antigens, protozoan antigens, helminth antigens, and tumor antigens.

In some embodiments, where an antigen is co-administered with an immunomodulatory nucleic acid, the antigen(s) is administered in the same formulation as the immunostimulatory nucleic acid. In some of these embodiments, the antigen is administered as the antigen per se. In other embodiments, e.g., where the antigen is a protein antigen, the antigen is encoded by a polynucleotide. In some of these embodiments, the polynucleotide that comprises a nucleotide sequence that encodes the antigen also comprises the immunomodulatory nucleic acid. In some of these embodiments, the polynucleotide that encodes the antigen is physically separate from the immunomodulatory nucleic acid.

In some embodiments, where an antigen is co-administered with an immunomodulatory nucleic acid, the antigen(s) is administered in a separate formulation from the composition comprising the immunostimulatory nucleic acid. In some of these embodiments, the antigen is administered as the antigen per se. In other embodiments, e.g., where the antigen is a protein antigen, the antigen is encoded by a polynucleotide. In some of these embodiments, the polynucleotide that comprises a nucleotide sequence that encodes the antigen also comprises the immunomodulatory nucleic acid. In some of these embodiments, the polynucleotide that encodes the antigen is physically separate from the immunomodulatory nucleic acid.

Where the antigen is co-administered with an immunomodulatory nucleic acid, and the antigen(s) is administered in a separate formulation from the composition comprising the immunostimulatory nucleic acid, in some embodiments, the separate formulations are administered substantially simultaneously. For example, in some embodiments, a composition comprising the antigen is administered within about 2 hours, about 1 hour, about 45 minutes, about 30 minutes, about 15 minutes, about 10 minutes, about 5 minutes, or less, of the composition comprising the immunomodulatory nucleic acid.

Exemplary Antigens

Tumor-specific antigens include, but are not limited to, any of the various MAGEs (Melanoma-Associated Antigen E), including MAGE 1 (e.g., GenBank Accession No. M77481), MAGE 2 (e.g., GenBank Accession No. U03735), MAGE 3, MAGE 4, etc.; any of the various tyrosinases; mutant ras; mutant p53 (e.g., GenBank Accession No. X54156 and AA494311); and p97 melanoma antigen (e.g., GenBank Accession No. M12154). Other tumor-specific antigens include the Ras peptide and p53 peptide associated with advanced cancers, the HPV 16/18 and E6/E7 antigens associated with cervical cancers, MUC1-KLH antigen associated with breast carcinoma (e.g., GenBank Accession No. J03651), CEA (carcinoembryonic antigen) associated with colorectal cancer (e.g., GenBank Accession No. X98311), gp100 (e.g., GenBank Accession No. S73003) or MART1 antigens associated with melanoma, and the PSA antigen associated with prostate cancer (e.g., GenBank Accession No. X14810). The p53 gene sequence is known (See e.g., Harris et al. (1986) Mol. Cell. Biol., 6:4650-4656) and is deposited with GenBank under Accession No. M14694. Cancers include, but are not limited to, carcinomas, lymphomas, leukemias, and sarcomas.

Suitable viral antigens include those derived from known causative agents responsible for diseases, including, but not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Japanese encephalitis (e.g., GenBank Accession No. E07883), dengue (e.g., GenBank Accession No. M24444), hantavirus, and HIV antigens (e.g., GenBank Accession No. U18552).

Suitable bacterial and parasitic antigens include those derived from known causative agents responsible for diseases including, but not limited to, diphtheria, pertussis (e.g., GenBank Accession No. M35274), tetanus (e.g., GenBank Accession No. M64353), tuberculosis, bacterial and fungal pneumonias (e.g., Haemophilus influenzae, Pneumocystis carinii, etc.), cholera, typhoid, plague, shigellosis, salmonellosis (e.g., GenBank Accession No. L03833), Legionnaire's Disease, Lyme disease (e.g., GenBank Accession No. U59487), malaria (e.g., GenBank Accession No. X53832), hookworm, onchocerciasis (e.g., GenBank Accession No. M27807), schistosomiasis (e.g., GenBank Accession No. L08198), trypanosomiasis, leshmaniasis, giardiasis (e.g., GenBank Accession No. M33641), amoebiasis, filariasis (e.g., GenBank Accession No. J03266), borreliosis, and trichinosis.

Maintaining Suppression of a Th2 Response

In some embodiments, methods of maintaining stimulation of a Th1 response are provided. Where the method is maintaining a suppression of a Th2 immune response, and the Th2 immune response is an allergic response to an allergen, the interval between the first dose and the second dose, or between any two subsequent consecutive doses is determined in part by the severity of the symptoms experienced by the individual. Such symptoms include, but are not limited to, sneezing, runny nose, watery eyes, allergic skin reactions (e.g., hives) and the like. For example, where such symptoms coincide with the presence in the environment of an allergen, such as a seasonal allergen, the interval may be determined, in part, by the abundance of the allergen in the individual's environment at a given time, where greater abundance of a given allergen indicates that the interval between consecutive doses should be decreased. The interval may also be determined by measuring an indicator of a Th2 immune response, as described above, e.g., by measuring the level of antigen-specific IgE in the individual, skin test (e.g., wheal and erythema test), and the like.

Thus, in some embodiments, the methods provide for maintaining suppression of an antigen-specific IgE level in an individual, involving administering consecutive doses of an immunomodulatory nucleic acid, as described above.

In some embodiments, the antigen to which the individual is sensitized is an allergen.

Allergens to which a person is sensitized include, but are not limited to, pollen, a mold spore allergen, a plant allergen, a non-human animal allergen, a human allergen, an insect allergen, a bacterial allergen, a viral allergen, a food allergen, an industrial chemical allergen, an aeroallergen (e.g., airborne pollen, airborne fungal spores, and the like), and a drug allergen.

In the context of this invention, the term “allergen” refers to an antigen that can trigger an allergic response which is mediated by IgE antibody. The method and compositions of this invention extend to a broad class of such allergens and fragments of allergens or haptens acting as allergens. These can include all the specific allergens that can cause an IgE-mediated response in allergic subjects. This invention is therefore useful for the treatment of allergic diseases in humans, other primates, and mammalian subjects, such as dogs, cats, and horses. Allergic diseases that are amenable to treatment using the compositions and methods of the instant invention include, but are not limited to, allergic diseases due to IgE; allergic rhinitis (hay fever); allergic asthma; atopic dermatitis; anaphylaxis; food allergy; drug allergy; urticaria (hives); angioedema; and allergic conjunctivitis.

Allergens include, but are not limited to, environmental aeroallergens; weed pollen allergens; grass pollen allergens; tree pollen allergens; house dust mite allergens; storage mite allergens; mold spore allergens; animal allergens (examples by species—cat, dog, guinea pig, hamster, gerbil, rat, mouse); animal allergens (examples by source—epithelial, salivary, urinary proteins); food allergens, including but not limited to the following common examples: crustaceans, nuts, such as peanuts, and citrus fruits; insect allergens (other than mites listed above); venoms, including, but not limited to, hymenoptera, yellow jacket, honey bee, wasp, hornet, and fire ant venoms; other environmental insect allergens from cockroaches, fleas, mosquitoes, etc.; bacteria such as streptococcal antigens; parasites such as ascaris antigen; viral antigens; drug allergens, such as antibiotics, e.g., penicillins and related compounds; other antibiotics; whole proteins such as hormones (e.g., insulin), enzymes (e.g., streptokinase); all drugs and their metabolites capable of acting as incomplete antigens or haptens; industrial chemicals and metabolites capable of acting as haptens and stimulating the immune system, including the following non-limiting examples: the acid anhydrides (such as trimellitic anhydride) and the isocyanates (such as toluene diisocyanate); occupational allergens such as flour in Baker's asthma, castor bean, coffee bean, and industrial chemicals described above.

Maintaining a Th1 Response

In some embodiments, methods of maintaining stimulation of a Th1 response are provided. In some embodiments, maintenance of a Th1 response includes maintaining stimulation of antigen-specific cytotoxic T lymphocytes (CTL). Stimulating production of antigen-specific CTL is useful in treating infection with an intracellular pathogen, and in treating cancer.

In some embodiments, the invention provides methods for inducing and maintaining protective immunity in a mammalian host to an intracellular pathogen, involving administering a composition comprising first dose and at least a second dose of an immunomodulatory nucleic acid, and an antigen of the pathogen. The immunomodulatory nucleic acid and the antigen are administered in amounts sufficient to induce and maintain protective immunity to the intracellular pathogen. Whether a subject method is effective in inducing and maintaining protective immunity to an intracellular pathogen can be determined using any known method. For example, the number of pathogenic organisms (virus, protozoan, etc.) present in a biological sample (e.g., blood, a blood product, serum, particular cells likely to be infected, and the like) obtained from the individual can be measured; the presence and/or the amount of an antigen associated with a given intracellular pathogen in a biological sample (e.g., blood, a blood product, or any biological fluid), can be determined; and the number of CTL specific for a given antigen from a pathogenic organism can be measured.

In other embodiments, the invention provides methods for inducing and maintaining immunity in a mammalian host to a tumor antigen, involving administering a composition comprising first dose and at least a second dose of an immunomodulatory nucleic acid, and a tumor antigen. The immunomodulatory nucleic acid and the antigen are administered in amounts sufficient to induce and maintain immunity to the tumor antigen. Whether the method is effective in treating the cancer can be determined using any standard method, including, but not limited to, measuring tumor mass, measuring the number of tumor cells, measuring the number of tumor-specific CTL, and the like.

Therapeutic Effects of the Methods of the Invention

The present invention provides methods for modulating an immune response to an antigen. In some embodiments, the methods involve administering to an individual who is sensitized to an antigen an immunostimulatory nucleic acid molecule. In many embodiments, the immunostimulatory nucleic acid molecule does not encode the antigen to which the individual is sensitized. In some embodiments, the immunostimulatory nucleic acid molecule is administered without the antigen to which the individual is sensitized. In other embodiments, the immunomodulatory nucleic acid is administered along with antigen (either as the antigen itself, or encoded in a polynucleotide).

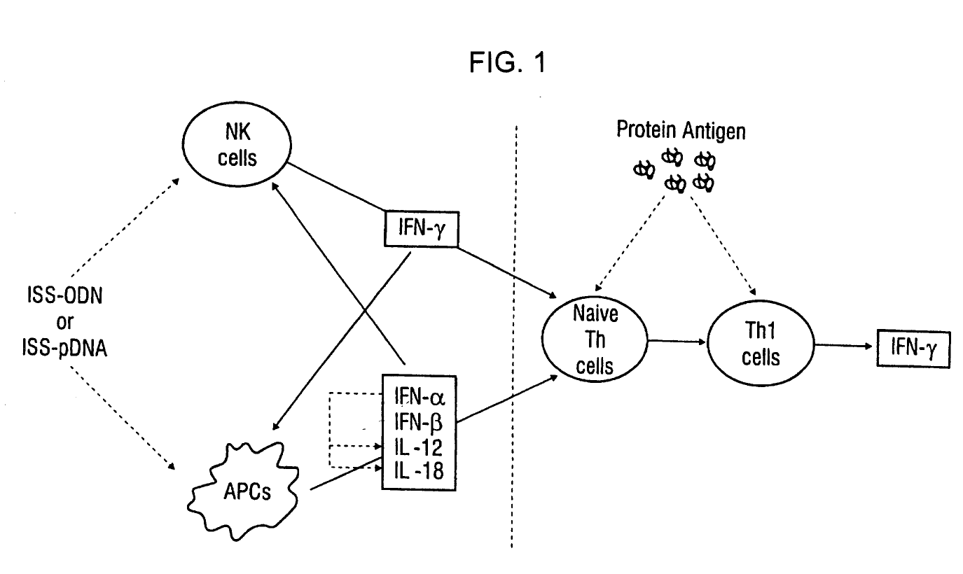

The main therapeutic goals which may be achieved through practice of the methods of the invention are treatment of inflammation and boosting of host immune responsiveness with a Th1 phenotype against a sensitizing antigen. Both goals are achieved by delivering ISS-ODN to an antigen-sensitized host; i.e., a mammal whose immune system has been primed to respond to challenge by a sensitizing antigen. For purposes of this disclosure, “sensitizing antigen” refers to an exogenous, immunogenic protein, peptide, glycoprotein, lipid or polysaccharide. For reference, a chart summarizing aspects of mammal antigen immunity is appended as FIG. 1.

The anti-inflammatory method of the invention is useful in suppressing the onset of, and in reducing, acute granulocyte-mediated inflammation in an antigen-sensitized host. Specifically, treatment of an antigen-sensitized (primed) host before subsequent antigen challenge suppresses antigen-stimulated infiltration of host tissue by granulocytes (especially, eosinophils and basophils). Similarly, treatment of an antigen-sensitized host on or after antigen challenge reduces antigen stimulated infiltration of host tissue by granulocytes. Advantageously, the anti-inflammatory impact of ISS-ODN delivered according to the invention is rapid, taking effect even before the ISS-ODN would be expected to impact the host's immune responsiveness to the sensitizing antigen. The invention therefore provides the host with fairly immediate protection against tissue damage from granulocyte-mediated inflammation.

For example, as shown by the data in Example II, antigen-sensitized animal models of allergic asthma treated with an immunomodulatory nucleic acid without concurrent antigen challenge experienced as much as a 90% reduction of eosinophil infiltration into respiratory tissue as compared to control animals and animals treated only with an inactive ISS mutant. Significantly, reduction of eosinophil infiltration in previously challenged mice, or suppression of eosinophil infiltration in primed, unchallenged mice, was obtained within as little as 24 hours of delivery of the ISS. The effect of the ISS on eosinophil infiltration is therefore independent of the later-developing host immune response to the sensitizing antigen. Being antigen independent, the ISS can be utilized as inflammation suppressors before antigen challenge or during a period when the risk of antigen challenge is present (e.g., during an allergy season). Importantly, as shown in Examples IV and VI, immunomodulatory nucleic acids can be used according to the invention to prevent inflammation or an immune response on subsequent antigen challenge in an antigen-primed host as well as to reduce inflammation or other antigen stimulated immune responses after antigen challenge.

Although the invention is not limited to any mechanism of action, it is probable that the antiinflammatory activity of ISS-ODN is at least in part a consequence of IL-5 suppression. However, suppression of granulocyte accumulation in host tissue is achieved more rapidly (within 24 hours) than immune activation of cytokine-secreting lymphocytes would be expected to occur. It is therefore also possible that ISS-ODN administered according to the invention physically interfere with granulocyte adhesion to endothelial, perhaps by blocking VCAM-1 endothelial receptors, theireosinophilic ligand (VLA-4) or by lysing granulocytes. Whatever the mechanism, ISS-ODN suppression of granulocyte accumulation according to the invention appears to be independent of ISS-ODN stimulation of the host immune system.

The immunotherapeutic method of the invention produces a vaccination-like immune response to challenge by a sensitizing antigen without concurrent exposure of the host to the antigen. Immune stimulation achieved through practice of the invention is comparable to the immune stimulation which occurs on vaccination of a host with a sensitizing antigen. Thus, the methods of the invention provides means to immunize a host against a sensitizing antigen without deliberate antigen challenge.

Advantageously, the immune response stimulated according to the invention differs from an immunization response in that the latter develops in a Th2 phenotype while the former develops in a Th1 phenotype. In this regard, it is helpful to recall that CD4+ lymphocytes generally fall into one of two distinct subsets; i.e., the Th1 and Th2 cells. Th1 cells principally secrete IL-2, IFN-γ and TNF (the latter two of which mediate macrophage activation and delayed type hypersensitivity) while Th2 cells principally secrete IL-4 (which stimulates production of IgE antibodies), IL-5 (which stimulates granulocyte infiltration of tissue), IL-6 and IL-10. These CD4+ subsets exert a negative influence on one another; i.e., secretion of Th1 lymphokines inhibits secretion of Th2 lymphokines and vice versa.

Factors believed to favor Th1 activation resemble those induced by viral infection and include intracellular pathogens, exposure to IFN-α, IFN-β, IFNγ, IL-12 and IL-18 and exposure to low doses of antigen. Th1 type immune responses also predominate in autoimmune disease. Factors believed to favor Th2 activation include exposure to IL-4 and IL-10, APC activity on the part of B lymphocytes and high doses of antigen. Active Th1 cells enhance cellular immunity and are therefore of particular value in responding to intracellular infections, while active Th2 cells enhance antibody production and are therefore of value in responding to extracellular infections (at the risk of anaphylactic events associated with IL-4 stimulated induction of IgE antibody production). Thus, the ability to shift host immune responses from the Th1 to the Th2 repertoire and vice versa has substantial clinical significance for controlling host immunity against antigen challenge (e.g., in infectious and allergic conditions).

To that end, the methods of the invention shift the host immune response to a sensitizing antigen toward a Th1 phenotype (Example IV). Consequently, antigen-stimulated/Th2 associated IL-4, IL-5 and IL-10 secretion (Example VI), IL-5 stimulated granulocyte infiltration of antigen-sensitized tissue (Examples II and III) and IL-4 stimulated production of IgE (Example V) are suppressed, thereby reducing the host's risk of prolonged allergic inflammation and minimizing the risk of antigen-induced anaphylaxis. Although the invention is not limited to any particular mechanism of action, it is conceivable that ISS-ODN facilitate uptake of exogenous antigen by antigen presenting cells for presentation through host MHC Class I processing pathways. Whatever the mechanism of action, use of ISS-ODN to boost the host's immune responsiveness to a sensitizing antigen and shift the immune response toward a Th1 phenotype avoids the risk of immunization-induced anaphylaxis, suppresses IgE production in response to a sensitizing antigen and eliminates the need to identify the sensitizing antigen for use in immunization.

With reference to the invention, ISS-ODN mediated “reduction of inflammation” (in a primed, antigen-challenged host), “prevention of inflammation” (in a primed host before antigen challenge) and “boosting of immune responsiveness in a Th1 phenotype” in an ISS-ODN treated host are evidenced by any of the following events:

(1) a reduction in levels of IL-4 measured before and after antigen-challenge; or detection of lower (or even absent) levels of IL-4 in a treated host as compared to an antigen primed, or primed and challenged, control;

(2) an increase in levels of IL-12, IL-18 and/or IFN-α, IFN-β, and IFN-γ before and after antigen challenge; or detection of higher levels of IL 12, IL-18 and/or IFN-α, IFN-β, and IFN-γ in an ISS-ODN treated host as compared to an antigen-primed or, primed and challenged, control;

(3) IgG2a antibody production in a treated host; or

(4) a reduction in levels of antigen-specific IgE as measured before and after antigen challenge; or detection of lower (or even absent) levels of antigen-specific IgE in an ISS-ODN treated host as compared to an antigen-primed, or primed and challenged, control.

Also, with respect to reduction and prevention of inflammation in particular, an especially meaningful indicia of the efficacy of the inventive method in a treated host is:

(5) a reduction in granulocyte counts (e.g., ofeosinophils or basophils, depending on which cell type is most involved in the condition affecting the host) in inflammatory infiltrate of an affected host tissue as measured in an antigen-challenged host before and after ISS-ODN administration, or detection of lower (or even absent) levels of eosinophil or basophil counts in a treated host as compared to an antigen-primed, or primed and challenged, control.

Exemplary methods for determining such values are described further in the Examples.

Nucleic Acid Molecules Comprising Immunomodulatory Nucleic Acid Molecule

Immunomodulatory nucleic acid molecules are polynucleotides that modulate activity of immune cells, especially immune cell activity associated with a type-1 (Th1-mediated) or type-1 like immune response.

Nucleic acid molecules comprising an immunomodulatory nucleic acid molecule which are suitable for use in the methods of the invention include an oligonucleotide, which can be a part of a larger nucleotide construct such as a plasmid. The term “polynucleotide” therefore includes oligonucleotides, modified oligonucleotides and oligonucleosides, alone or as part of a larger construct. The polynucleotide can be single-stranded DNA (ssDNA), double-stranded DNA (dsDNA), single-stranded RNA (ssRNA) or double-stranded RNA (dsRNA). The polynucleotide portion can be linearly or circularly configured, or the oligonucleotide portion can contain both linear and circular segments. Immunomodulatory nucleic acid molecules also encompasses crude, detoxified bacterial (e.g., mycobacterial) RNA or DNA, as well as ISS-enriched plasmids. “ISS-enriched plasmid” as used herein is meant to refer to a linear or circular plasmid that comprises or is engineered to comprise a greater number of CpG motifs than normally found in mammalian DNA. Exemplary ISS-enriched plasmids are described in, for example, Roman et al. (1997) Nat. Med. 3(8):849-54. Modifications of oligonucleotides include, but are not limited to, modifications of the 3′OH or 5′OH group, modifications of the nucleotide base, modifications of the sugar component, and modifications of the phosphate group.

The immunomodulatory nucleic acid molecule can comprise ribonucleotides (containing ribose as the only or principal sugar component), deoxyribonucleotides (containing deoxyribose as the principal sugar component), or in accordance with the established state-of-the-art, modified sugars or sugar analogs may be incorporated in the oligonucleotide of the present invention. Examples of a sugar moiety that can be used include, in addition to ribose and deoxyribose, pentose, deoxypentose, hexose, deoxyhexose, glucose, arabinose, xylose, lyxose, and a sugar “analog” cyclopentyl group. The sugar may be in pyranosyl or in a furanosyl form. In the modified oligonucleotides of the present invention, the sugar moiety is preferably the furanoside of ribose, deoxyribose, arabinose or 2′-O-methylribose, and the sugar may be attached to the respective heterocyclic bases either in or anomeric configuration.

An immunomodulatory nucleic acid molecule may comprise at least one nucleoside comprising an L-sugar. The L-sugar may be deoxyribose, ribose, pentose, deoxypentose, hexose, deoxyhexose, glucose, galactose, arabinose, xylose, lyxose, or a sugar “analog” cyclopentyl group. The L-sugar may be in pyranosyl or furanosyl form.

The phosphorous derivative (or modified phosphate group) that can be attached to the sugar or sugar analog moiety in the modified oligonucleotides of the present invention can be a monophosphate, diphosphate, triphosphate, alkylphosphate, alkanephosphate, phosphoronthioate, phosphorodithioate or the like. The heterocyclic bases, or nucleic acid bases that are incorporated in the oligonucleotide base of the ISS can be the naturally occurring principal purine and pyrimidine bases, (namely uracil or thymine, cytosine, adenine and guanine, as mentioned above), as well as naturally occurring and synthetic modifications of said principal bases. Those skilled in the art will recognize that a large number of “synthetic” non-natural nucleosides comprising various heterocyclic bases and various sugar moieties (and sugar analogs) are available, and that the immunomodulatory nucleic acid molecule can include one or several heterocyclic bases other than the principal five base components of naturally occurring nucleic acids. Preferably, however, the heterocyclic base in the ISS is selected from uracil-5-yl, cytosin-5-yl, adenin-7-yl, adenin-8-yl, guanin-7-yl, guanin-8-yl, 4-aminopyrrolo[2,3-d]pyrimidin-5-yl, 2-amino-4-oxopyrolo[2,3-d]pyrimidin-5-yl, 2-amino-4-oxopyrrolo[2,3-d]pyrimidin-3-yl groups, where the purines are attached to the sugar moiety of the oligonucleotides via the 9-position, the pyrimidines via the 1-position, the pyrrolopyrimidines via the 7-position and the pyrazolopyrimidines via the 1-position.

Structurally, the root oligonucleotide of the immunomodulatory nucleic acid molecule is a non-coding sequence that can include at least one unmethylated CpG motif. The relative position of any CpG sequence in ISS with immunomodulatory activity in certain mammalian species (e.g., rodents) is 5′-CG-3′ (i.e., the C is in the 5′ position with respect to the G in the 3′position).

Immunomodulatory nucleic acid molecules generally do not provide for, nor is there any requirement that they provide for, expression of any amino acid sequence encoded by the polynucleotide, and thus the sequence of a immunomodulatory nucleic acid molecule may be, and generally is, non-coding. Immunomodulatory nucleic acid molecules may comprise a linear double or single-stranded molecule, a circular molecule, or can comprise both linear and circular segments. Immunomodulatory nucleic acid molecules may be single-stranded, or may be completely or partially double-stranded.

In some embodiments, an immunomodulatory nucleic acid molecule is an oligonucleotide, e.g., consists of a sequence of from about 6 to about 200, from about 10 to about 100, from about 12 to about 50, or from about 15 to about 25, nucleotides in length.

Exemplary consensus CpG motifs of immunomodulatory nucleic acid molecules useful in the invention include, but are not necessarily limited to:

5′-Purine-Purine-C-G-Pyrimidine-Pyrimidine-3′, in which the immunomodulatory nucleic acid molecule comprises a CpG motif flanked by at least two purine nucleotides (e.g., GG, GA, AG, AA, II, etc.,) and at least two pyrimidine nucleotides (CC, TT, CT, TC, UU, etc.);

5′-Purine-TCG-Pyrimidine-Pyrimidine-3′;

5′-(TCG)n-3′, where n is any integer that is 1 or greater, e.g., to provide a poly-TCG immunomodulatory nucleic acid molecule (e.g., where n=3, the polynucleotide comprises the sequence 5′-TCGTCGTCG-3′); and

5′-Purine-Purine-CG-Pyrimidine-Pyrimidine-CG-3′.

5′-Purine-TCG-Pyrimidine-Pyrimidine-CG-3′

Exemplary DNA-based immunomodulatory nucleic acid molecules useful in the invention include, but are not necessarily limited to, polynucleotides comprising the following nucleotide sequences:

AACGCC, AACGCT, AACGTC, AACGTT;
AGCGCC, AGCGCT, AGCGTC, AGCGTT;
GACGCC, GACGCT, GACGTC, GACGTT;
GGCGCC, GGCGCT, GGCGTC, GGCGTT;
ATCGCC, ATCGCT, ATCGTC, ATCGTT;
GTCGCC, GTCGCT, GTCGTC, GTCGTT; and
TCGTCQG, and TCGTCGTCG

Octameric sequences are generally the above-mentioned hexameric sequences, with an additional 3′ CG. Exemplary DNA-based immunomodulatory nucleic acid molecules useful in the invention include, but are not necessarily limited to, polynucleotides comprising the following octameric nucleotide sequences:

AACGCCCG, AACGCTCG, AACGTCCG, AACGTTCG;
AGCGCCCG, AGCGCTCG, AGCGTCCG, AGCGTTCG;
GACGCCCG, GACGCTCG, GACGTCCG, GACGTTCG;
GGCGCCCG, GGCGCTCG, GGCGTCCG, GGCGTTCG;
ATCGCCCG, ATCGCTCG, ATCGTCCG, ATCGTTCG;
GTCGCCCG, GTCGCTCG, GTCGTCCG, and GTCGTTCG.

Immunomodulatory nucleic acid molecules useful in the invention can comprise one or more of any of the above CpG motifs. For example, immunomodulatory nucleic acid molecules useful in the invention can comprise a single instance or multiple instances (e.g., 2, 3, 5 or more) of the same CpG motif. Alternatively, the immunomodulatory nucleic acid molecules can comprises multiple CpG motifs (e.g., 2, 3, 5 or more) where at least two of the multiple CpG motifs have different consensus sequences, or where all CpG motifs in the immunomodulatory nucleic acid molecules have different consensus sequences.

A non-limiting example of an immunomodulatory nucleic acid molecule is one with the sequence 5′-TGACTGTGAACGTTCGAGATGA-3′ (SEQ ID NO:1). An example of a control nucleic acid molecule is one having the sequence 5′-TGACTGTGAAgGTTCGAGATGA-3′ (SEQ ID NO:2), which differs from SEQ ID NO:1 at the nucleotide indicated in lower case type.

Immunomodulatory nucleic acid molecules useful in the invention may or may not include palindromic regions. If present, a palindrome may extend only to a CpG motif, if present, in the core hexamer or octamer sequence, or may encompass more of the hexamer or octamer sequence as well as flanking nucleotide sequences.

The core hexamer structure of the foregoing immunomodulatory nucleic acid molecules can be flanked upstream and/or downstream by any number or composition of nucleotides or nucleosides. However, ISS are at least 6 bases in length, and preferably are between 6 and 200 bases in length, to enhance uptake of the immunomodulatory nucleic acid molecule into target tissues.

In particular, immunomodulatory nucleic acid molecules useful in the invention include those that have hexameric nucleotide sequences having “CpG” motifs. Although DNA sequences are generally preferred, RNA immunomodulatory nucleic acid molecules can be used, with inosine and/or uracil substitutions for nucleotides in the hexamer sequences.

Modifications

Immunomodulatory nucleic acid molecules can be modified in a variety of ways. For example, the immunomodulatory nucleic acid molecules can comprise backbone phosphate group modifications (e.g., methylphosphonate, phosphorothioate, phosphoroamidate and phosphorodithioate internucleotide linkages), which modifications can, for example, enhance stability of the immunomodulatory nucleic acid molecule in vivo, making them particularly useful in therapeutic applications. A particularly useful phosphate group modification is the conversion to the phosphorothioate or phosphorodithioate forms of an immunomodulatory nucleic acid molecule. Phosphorothioates and phosphorodithioates are more resistant to degradation in vivo than their unmodified oligonucleotide counterparts, increasing the half-lives of the immunomodulatory nucleic acid molecules and making them more available to the subject being treated.

Other modified immunomodulatory nucleic acid molecules encompassed by the present invention include immunomodulatory nucleic acid molecules having modifications at the 5′ end, the 3′ end, or both the 5′ and 3′ ends. For example, the 5′ and/or 3′ end can be covalently or non-covalently conjugated to a molecule (either nucleic acid, non-nucleic acid, or both) to, for example, increase the bio-availability of the immunomodulatory nucleic acid molecules, increase the efficiency of uptake where desirable, facilitate delivery to cells of interest, and the like. Exemplary molecules for conjugation to the immunomodulatory nucleic acid molecules include, but are not necessarily limited to, cholesterol, phospholipids, fatty acids, sterols, oligosaccharides, polypeptides (e.g., immunoglobulins), peptides, antigens (e.g., peptides, small molecules, etc.), linear or circular nucleic acid molecules (e.g., a plasmid), and the like.

Additional immunomodulatory nucleic acid conjugates, and methods for making same, include conjugates with immunomodulatory nucleic acid and an antigen. In this context “conjugates” includes both covalently linked molecules, as well as molecules that are proximately associated, e.g., the immunomodulatory nucleic acid and the antigen are in close proximity so as to provide for enhanced effects of immunomodulatory nucleic acid and antigen combination relative to co-administered immunomodulatory nucleic acid and antigen. Immunomodulatory nucleic acid conjugates are known in the art and described in, for example, WO 98/16427 and WO 98/55495. Thus, the term “immunomodulatory nucleic acid molecule” includes conjugates comprising an immunomodulatory nucleic acid molecule.

Preparation of Immunomodulatory Nucleic Acid Molecules

Immunomodulatory nucleic acid molecules can be synthesized using techniques and nucleic acid synthesis equipment well known in the art (see, e.g., Ausubel et al. Current Protocols in Molecular Biology, (Wiley Intersicence, 1989); Maniatis et al. Molecular Cloning: A Laboratory Manual (Cold Spring Harbor Laboratories, New York, 1982); and U.S. Pat. Nos. 4,458,066; and 4,650,675. Individual polynucleotide fragments can be ligated with a ligase such as T4 DNA or RNA ligase as described in, e.g., U.S. Pat. No. 5,124,246. Oligonucleotide degradation can be accomplished through exposure to a nuclease, see, e.g., U.S. Pat. No. 4,650,675. As noted above, since the immunomodulatory nucleic acid molecules need not provide for expression of any encoded amino acid sequence, the invention does not require that the immunomodulatory nucleic acid molecules be operably linked to a promoter or otherwise provide for expression of a coding sequence.

Alternatively, immunomodulatory nucleic acid molecules can be isolated from microbial species (e.g., mycobacteria) using techniques well known in the art such as nucleic acid hybridization, amplification (e.g., by PCR), and the like. Isolated immunomodulatory nucleic acid molecules can be purified to a substantially pure state, e.g., free of endogenous contaminants, e.g., lipopolysaccharides. Immunomodulatory nucleic acid molecules isolated as part of a larger polynucleotide can be reduced to the desired length by techniques well known in the art, such as endonuclease digestion. Other techniques suitable for isolation, purification, and production of polynucleotides to obtain ISS will be readily apparent to the ordinarily skilled artisan in the relevant field.

Circular immunomodulatory nucleic acid molecules can also be synthesized through recombinant methods or chemically synthesized. Where circular immunomodulatory nucleic acid molecules are obtained through isolation or recombinant methods, the immunomodulatory nucleic acid molecule can be provided as a plasmid. Chemical synthesis of smaller circular oligonucleotides can be performed using methods known in the art (see, e.g., Gao et al. (1995) Nucl. Acids. Res. 23:2025-9; Wang et al., (1994) Nucl. Acids Res. 22:2326-33).

Where the immunomodulatory nucleic acid molecule comprises a modified oligonucleotide, the modified oligonucleotides can be synthesized using standard chemical techniques. For example, solid-support based construction of methylphosphonates has been described in Agrawal et al. Tet. Lett. 28:3539-42. Synthesis of other phosphorous-based modified oligonucleotides, such as phosphotriesters (see, e.g., Miller et al. (1971) J. Am Chem Soc. 93:6657-65), phosphoramidates (e.g., Jager et al. (1988) Biochem. 27:7237-46), and phosphorodithioates (e.g., U.S. Pat. No. 5,453,496) is known in the art. Other non-phosphorous-based modified oligonucleotides can also be used (e.g., Stirchak et al. (1989) Nucl. Acids. Res. 17:6129-41).

Preparation of base-modified nucleosides, and the synthesis of modified oligonucleotides using such base-modified nucleosides as precursors is well known in the art, see, e.g., U.S. Pat. Nos. 4,910,300; 4,948,882; and 5,093,232. These base-modified nucleosides have been designed so that they can be incorporated by chemical synthesis into either terminal or internal positions of an oligonucleotide. Nucleosides modified in their sugar moiety have also been described (see, e.g., U.S. Pat. Nos. 4,849,513; 5,015,733; 5,118,800; and 5,118,802).

Techniques for making phosphate group modifications to oligonucleotides are known in the art. Briefly, an intermediate phosphate triester for the target oligonucleotide product is prepared and oxidized to the naturally-occurring phosphate triester with aqueous iodine or other agents, such as anhydrous amines. The resulting oligonucleotide phosphoramidates can be treated with sulfur to yield phosphorothioates. The same general technique (without the sulfur treatment step) can be used to produced methylphosphoamidites from methylphosphonates. Techniques for phosphate group modification are well known and are described in, for example, U.S. Pat. Nos. 4,425,732; 4,458,066; 5,218,103; and 5,453,496.

Identification of Immunomodulatory Nucleic Acid Molecules

Confirmation that a particular compound has the properties of an immunomodulatory nucleic acid molecule useful in the invention can be obtained by evaluating whether the immunomodulatory nucleic acid molecule elicits the appropriate cytokine secretion patterns, e.g., a cytokine secretion pattern associated with a type-1 immune response; inhibits intracellular pathogen replication, e.g., inhibits intracellular growth of intracellular pathogens either in vitro or in vivo; and/or modulates intracellular availability of cellular products necessary for growth and/or reproduction of the intracellular pathogen, e.g., reduces intracellular levels of L-tryptophan, for example, by inducing expression of indoleamine 2,3-dioxygenase (IDO) in a cell. ISS delivered with an antigen also induces activity of cytotoxic T cells and acts as a very strong mucosal adjuvant (see, e.g., Horner (1998) Cell. Immunol. 190:77-82). As noted above, immunomodulatory nucleic acid molecules of interest in the methods of the invention are those that elicit a Th1-mediated response.

In general, helper T (Th) cells are divided into broad groups based on their specific profiles of cytokine production: Th1, Th2, and Th0. “Th1” cells are T lymphocytes that release predominantly the cytokines IL-2 and IFN-γ, which cytokines in turn promote T cell proliferation, facilitate macrophage activation, and enhance the cytolytic activity of natural killer (NK) cells and antigen-specific cytotoxic T cells (CTL). In contrast, the cytokines predominantly released by Th2 cells are IL-4, IL-5, and IL-10. IL-4 and IL-5 are known to mediate antibody isotype switching towards IgE or IgA response, and promote eosinophil recruitment, skewing the immune system toward an “allergic” type of response. Th0 cells release a set of cytokines with characteristics of both Th1-type and Th2-type responses. While the categorization of T cells as Th1, TH2, or Th0 is helpful in describing the differences in immune response, it should be understood that it is more accurate to view the T cells and the responses they mediate as forming a continuum, with Th1 and Th2 cells at opposite ends of the scale, and Th0 cells providing the middle of the spectrum. Therefore, it should be understood that the use of these terms herein is only to describe the predominant nature of the immune response elicited, and is not meant to be limiting to an immune response that is only of the type indicated. Thus, for example, reference to a “type-1” or “Th1” immune response is not meant to exclude the presence of a “type-2” or “Th2” immune response, and vice versa.

Details of in vitro and in vivo techniques useful for evaluation of production of cytokines associated with a type-1 or type2 response, as well as for evaluation of antibody production, are well known in the art. Likewise, methods for evaluating the ability of candidate ISS to inhibit intracellular pathogen growth are also well known in the art, and are further exemplified in the Examples below.

Administration of Immunomodulatory Nucleic Acid Molecules

Immunomodulatory nucleic acid molecules are administered to an individual using any available method and route suitable for drug delivery, including in vivo and ex vivo methods, as well as systemic, mucosal, and localized routes of administration.

Conventional and pharmaceutically acceptable routes of administration include intranasal, intramuscular, intratracheal, subcutaneous, intradermal, topical application, intravenous, rectal, nasal, oral and other parenteral routes of administration. Routes of administration may be combined, if desired, or adjusted depending upon the immunomodulatory nucleic acid molecule and/or the desired effect on the immune response. The immunomodulatory nucleic acid composition can be administered in a single dose or in multiple doses, and may encompass administration of booster doses, to elicit and/or maintain the desired effect on the immune response.

Immunomodulatory nucleic acid molecules can be administered to a subject using any available conventional methods and routes suitable for delivery of conventional drugs, including systemic or localized routes. Methods and localized routes that further facilitate production of a type-1 or type-1-like response activity of the immunomodulatory nucleic acid molecules are of interest in the invention, and may be preferred over systemic routes of administration, both for the immediacy of therapeutic effect and avoidance of in vivo degradation of the administered immunomodulatory nucleic acid molecules. In general, routes of administration contemplated by the invention include, but are not necessarily limited to, enteral, parenteral, or inhalational routes. Inhalational routes may be preferred in cases of pulmonary involvement, particularly in view of the activity of immunomodulatory nucleic acid molecules as a mucosal adjuvant.

Inhalational routes of administration (e.g., intranasal, intrapulmonary, and the like) are particularly useful in stimulating an immune response for prevention or treatment of intracellular pathogen infections of the respiratory tract. Such means include inhalation of aerosol suspensions or insufflation of the polynucleotide compositions of the invention. Nebulizer devices, nasal sprays, nasal inhalation devices, metered dose inhalers, and the like suitable for delivery of polynucleotide compositions to the nasal mucosa, trachea and bronchioli are well-known in the art and will therefore not be described in detail here. For general review in regard to intranasal drug delivery, see, e.g., Chien, Novel Drug Delivery Systems, Ch. 5 (Marcel Dekker, 1992).

Delivery by inhalation can be via insufflation of an aerosolized formulation comprising an immunostimulatory nucleic acid molecule. As used herein, the term “aerosol” is used in its conventional sense as referring to very fine liquid or solid particles carries by a propellant gas under pressure to a site of therapeutic application. The term “liquid formulation for delivery to respiratory tissue” and the like, as used herein, describe compositions comprising an immunostimulatory nucleic acid molecule with a pharmaceutically acceptable carrier in flowable liquid form. Such formulations, when used for delivery to a respiratory tissue, are generally solutions, e.g. aqueous solutions, ethanoic solutions, aqueous/ethanoic solutions, saline solutions and colloidal suspensions. In general, aerosolized particles for respiratory delivery must have a diameter of 12 microns or less. However, the preferred particle size varies with the site targeted (e.g, delivery targeted to the bronchi, bronchia, bronchioles, alveoli, or circulatory system). For example, topical lung treatment can be accomplished with particles having a diameter in the range of 1.0 to 12.0 microns. Effective systemic treatment requires particles having a smaller diameter, generally in the range of 0.5 to 6.0 microns. Thus, in some embodiments, at least about 40%, at least about 50%, at least about 60%, at least about 70%, at least about 80%, at least about 90%, or more, of an aerosolized formulation comprising immunostimulatory nucleic acid molecules for delivery to a respiratory tissue is composed of particles in the range of from about 0.5 to about 12 micrometers, from about 0.5 to about 6 micrometers, or from about 1.0 to about 12 micrometers.

The formulation for delivery to a respiratory tissue may be provided in a container suitable for delivery of aerosolized formulations. U.S. Pat. Nos. 5,544,646; 5,709,202; 5,497,763; 5,544,646; 5,718,222; 5,660,166; 5,823,178; 5,829,435; and 5,906,202 describe devices and methods useful in the generation of aerosols suitable for drug delivery, any of which can be used in the present invention for delivering a formulation comprising an immunostimulatory nucleic acid molecule to a respiratory tissue. In some embodiments, the invention provides a container, which may be a disposable container, having at least one wall that is collapsible upon application of a force, wherein at least one wall has an opening. A porous membrane having pores in a range of from about 0.25 microns to about 6 microns covers the opening. The container comprises a flowable liquid formulation comprising an immunostimulatory nucleic acid molecule. Upon application of a force, the flowable liquid formulation is forced through the pores in the membrane and is aerosolized. The container may be provided in any known configuration, e.g., a blister pack. The container may be provided together with an aerosol delivery device, such that the aerosolized formulation exits the container and proceeds through a channel in an aerosol delivery device and into the respiratory tract of an individual.

When a pharmaceutical aerosol is employed in this invention, the aerosol contains a immunostimulatory nucleic acid molecule, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Examples of suitable propellants include, but is not limited to, hydrocarbons or other suitable gas. In the case of the pressurized aerosol, the dosage unit may be determined by providing a value to deliver a metered amount.

Administration of formulation comprising an immunostimulatory nucleic acid molecule can also be carried out with a nebulizer, which is an instrument that generates very fine liquid particles of substantially uniform size in a gas. Preferably, a liquid containing the immunostimulatory nucleic acid molecule is dispersed as droplets. The small droplets can be carried by a current of air through an outlet tube of the nebulizer. The resulting mist penetrates into the respiratory tract of the patient.

A powder composition containing immunostimulatory nucleic acid molecules, with or without a lubricant, carrier, or propellant, can be administered to a mammal in need of therapy. This embodiment of the invention can be carried out with a conventional device for administering a powder pharmaceutical composition by inhalation. For example, a powder mixture of the compound and a suitable powder base such as lactose or starch may be presented in unit dosage form in for example capsular or cartridges, e.g. gelatin, or blister packs, from which the powder may be administered with the aid of an inhaler.

Combination therapies may be used to treat a respiratory condition and to modulate an immune response, as described herein. In particular, immunostimulatory nucleic acid molecules may be combined with conventional therapeutic agents for treating various respiratory diseases such as asthma, bronchitis, etc. Therapeutic agents for treating respiratory diseases which may be administered in combination with an immunostimulatory nucleic acid molecule of the invention include, but are not limited to beta adrenergics which include bronchodilators including albuterol, isoproterenol sulfate, metaproterenol sulfate, terbutaline sulfate, pirbuterol acetate and salmeterol formotorol; steroids including beclomethasone dipropionate, flunisolide, fluticasone, budesonide and triamcinolone acetonide. Anti-inflammatory drugs used in connection with the treatment of respiratory diseases include steroids such as beclomethasone dipropionate, triamcinolone acetonide, flunisolide and fluticasone. Other anti-inflammatory drugs include cromoglycates such as cromolyn sodium.

Other respiratory drugs which would qualify as bronchodilators include anticholenergics including ipratropium bromide. Anti-histamines include, but are not limited to, diphenhydramine, carbinoxamine, clemastine, dimenhydrinate, pryilamine, tripelennamine, chlorpheniramine, brompheniramine, hydroxyzine, cyclizine, meclizine, chlorcyclizine, promethazine, doxylamine, loratadine, and terfenadine. Particular anti-histamines include rhinolast (Astelin), claratyne (Claritin), claratyne D (Claritin D), telfast (Allegra), zyrtec, and beconase.

The present invention is intended to encompass the free acids, free bases, salts, amines and various hydrate forms including semi-hydrate forms of such respiratory drugs and is particularly directed towards pharmaceutically acceptable formulations of such drugs which are formulated in combination with pharmaceutically acceptable excipient materials generally known to those skilled in the art—in some embodiments without other additives such as preservatives. In some embodiments, drug formulations do not include additional components which have a significant effect on the overall formulation such as preservatives. Thus certain formulations consist essentially of pharmaceutically active drug and a pharmaceutically acceptable carrier (e.g., water and/or ethanol). However, if a drug is liquid without an excipient the formulation may consist essentially of the drug which has a sufficiently low viscosity that it can be aerosolized using a dispenser.

Administration by inhalation is preferred in some cases because smaller doses can be delivered locally to the specific cells (e.g., cells of respiratory tissue) which are most in need of treatment. By delivering smaller doses, any adverse side effects are eliminated or substantially reduced. By delivering directly to the cells which are most in need of treatment, the effect of the treatment will be realized more quickly.

There are several different types of inhalation methodologies which can be employed in connection with the present invention. Immunostimulatory nucleic acid molecules of the present invention can be formulated in basically three different types of formulations for inhalation. First, antagonists of the invention can be formulated with low boiling point propellants. Such formulations are generally administered by conventional meter dose inhalers (MDI's). However, conventional MDI's can be modified so as to increase the ability to obtain repeatable dosing by utilizing technology which measures the inspiratory volume and flow rate of the patient as discussed within U.S. Pat. Nos. 5,404,871 and 5,542,410.

Alternatively, immunostimulatory nucleic acid molecules of the present invention can be formulated in aqueous or ethanolic solutions and delivered by conventional nebulizers. However, more preferably, such solution formulations are aerosolized using devices and systems such as disclosed within U.S. Pat. Nos. 5,497,763; 5,544,646; 5,718,222; and 5,660,166.

In addition, immunostimulatory nucleic acid molecules of the present invention can be formulated into dry powder formulations. Such formulations can be administered by simply inhaling the dry powder formulation after creating an aerosol mist of the powder. Technology for carrying such out is described within U.S. Pat. No. 5,775,320 issued Jul. 7, 1998 and U.S. Pat. No. 5,740,794 issued Apr. 21, 1998.

With respect to each of the patents recited above, applicants point out that these patents cite other publications in intrapulmonary drug delivery and such publications can be referred to for specific methodology, devices and formulations which could be used in connection with the delivery of agonists of the present invention. Further, each of the patents are incorporated herein by reference in their entirety for purposes of disclosing formulations, devices, packaging and methodology for the delivery of immunostimulatory nucleic acid molecule formulations of the present invention.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of immunomodulatory nucleic acid molecules. Where systemic delivery is desired, administration typically involves invasive or systemically absorbed topical or mucosal administration of pharmaceutical preparations.

Immunomodulatory nucleic acid molecules can also be delivered to the subject by enteral administration. Enteral routes of administration include, but are not necessarily limited to, oral and rectal (e.g., using a suppository) delivery.

Methods of administration of immunomodulatory nucleic acid molecules through the skin or mucosa include, but are not necessarily limited to, topical application of a suitable pharmaceutical preparation, transdermal transmission, injection and epidermal administration. For transdermal transmission, absorption promoters or iontophoresis are suitable methods. For review regarding such methods, those of ordinary skill in the art may wish to consult Chien, supra at Ch. 7. Iontophoretic transmission may be accomplished using commercially available “patches” which deliver their product continuously via electric pulses through unbroken skin for periods of several days or more. An exemplary patch product for use in this method is the LECTRO PATCH™ (manufactured by General Medical Company, Los Angeles, Calif.) which electronically maintains reservoir electrodes at neutral pH and can be adapted to provide dosages of differing concentrations, to dose continuously and/or to dose periodically.

Epidermal administration can be accomplished by mechanically or chemically irritating the outermost layer of the epidermis sufficiently to provoke an immune response to the irritant. An exemplary device for use in epidermal administration employs a multiplicity of very narrow diameter, short tynes which can be used to scratch ISS coated onto the tynes into the skin. The device included in the MONO-VACC™ tuberculin test (manufactured by Pasteur Merieux, Lyon, France) is suitable for use in epidermal administration of immunomodulatory nucleic acid molecules.

The invention also contemplates opthalmic administration of immunomodulatory nucleic acid molecules, which generally involves invasive or topical application of a pharmaceutical preparation to the eye. Eye drops, topical creams and injectable liquids are all examples of suitable formulations for delivering drugs to the eye.

Immunomodulatory nucleic acid molecules can be administered to a subject prior to exposure to intracellular pathogen, after exposure to intracellular pathogen but prior to onset of disease symptoms associated with infection, or after intracellular pathogen infection or onset of disease symptoms. As such, immunomodulatory nucleic acids can be administered at any time after exposure to intracellular pathogen, but a first dose is usually administered about 8 hours, about 12 hours, about 24 hours, about 2 days, about 4 days, about 8 days, about 16 days, about 30 days or 1 month, about 2 months, about 4 months, about 8 months, or about 1 year after exposure to intracellular pathogen. As described in more detail herein, the invention also provides for administration of subsequent doses of immunomodulatory nucleic acid molecules.

Dosages

One particular advantage of the use of immunomodulatory nucleic acid molecules in the methods of the invention is that immunomodulatory nucleic acid molecules exert immunomodulatory activity even at relatively low dosages. Although the dosage used will vary depending on the clinical goals to be achieved, a suitable dosage range is one which provides up to about 1 μg, to about 1,000 μg, to about 10,000 μg, to about 25,000 μg or about 50,000 μg of ISS. Immunomodulatory nucleic acid molecules can be administered in a single dosage or several smaller dosages over time. Alternatively, a target dosage of ISS can be considered to be about 1-10 μM in a sample of host blood drawn within the first 24-48 hours after administration of ISS. Based on current studies, immunomodulatory nucleic acid molecules are believed to have little or no toxicity at these dosage levels.

It should be noted that the immunotherapeutic activity of immunomodulatory nucleic acid molecules in the invention is essentially dose-dependent. Therefore, to increase ISS potency by a magnitude of two, each single dose is doubled in concentration. Increased dosages may be needed to achieve the desired therapeutic goal. The invention thus contemplates administration of “booster” doses to provide and maintain an immune response effective to protect the subject from infection or to inhibit infection; to reduce the risk of the onset of disease or the severity of disease symptoms that may occur as a result of infection; to facilitate reduction of intracellular pathogen load; and/or to facilitate clearance of infecting intracellular pathogen from the subject (e.g., to facilitate clearance of organisms from the lungs). When multiple doses are administered, subsequent doses (“boosters”) are administered within about 16 weeks, about 12 weeks, about 8 weeks, about 6 weeks, about 4 weeks, about 2 weeks, about 1 week, about 5 days, about 72 hours, about 48 hours, about 24 hours, about 12 hours, about 8 hours, about 4 hours, or about 2 hours or less of the previous dose.

In view of the teaching provided by this disclosure, those of ordinary skill in the clinical arts will be familiar with, or can readily ascertain, suitable parameters for administration of ISS according to the invention.

Formulations

In general, immunomodulatory nucleic acid molecules are prepared in a pharmaceutically acceptable composition for delivery to a host. Pharmaceutically acceptable carriers preferred for use with the immunomodulatory nucleic acid of the invention may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition of ISS may also be lyophilized using means well known in the art, for subsequent reconstitution and use according to the invention. Also of interest are formulations for liposomal delivery, and formulations comprising microencapsulated immunomodulatory nucleic acid molecules.

In general, the pharmaceutical compositions can be prepared in various dosage forms, such as granules, tablets, pills, suppositories, capsules, suspensions, salves, lotions and the like. Pharmaceutical grade organic or inorganic carriers and/or diluents suitable for oral and topical use can be used to make up compositions comprising the therapeutically active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. Preservatives and other additives may also be present such as, for example, anti-pathogenic agents (e.g., antimicrobials, antibacterials, antivirals, antifungals, etc.), antioxidants, chelating agents, and inert gases and the like. In one embodiment, as discussed above, the immunomodulatory nucleic acid molecule formulation comprises an anti-pathogenic agent.

Exemplary anti-pathogenic agents include, but are not necessarily limited to, antibiotics, including antimicrobial agents (e.g., bacteriostatic and bacteriocidal agents (e.g., aminoglycosides, β-lactam antibiotics, cephalosporins, macrolides, penicillins, tetracyclines, quinolones, and the like ), antivirals (e.g., amprenavirs, acyclovirs, amantadines, virus penciclovirs, and the like), and the like), antifungals, (e.g., imidazoles, triazoles, allylamines, polyenes, and the like), as well as anti-parasitic agents (e.g., atovaquones, chloroquines, pyrimethamines, ivermectins, mefloquines, pentamidines, primaquines, and the like). In another embodiment, the anti-pathogenic agent is an anti-mycobacterial agent (e.g., clarithromycin; capreomycin sulfate; ethambutol HCl; isoniazid; aminosalicylic acid; rifapentine; PYRAZINAMIDE; rifampin; rifampin and isoniazid in combination; rifampin, isoniazid, and pyrazinamide in combination; cycloserine; streptomycin sulfate; ethionamide; and the like).

Immunomodulatory nucleic acid molecules can be administered in the absence of agents or compounds that might facilitate uptake by target cells (e.g., as a “naked” polynucleotide, e.g., a polynucleotide that is not encapsulated by a viral particle). Immunomodulatory nucleic acid molecules can also be administered with compounds that facilitate uptake of immunomodulatory nucleic acid molecules by target cells (e.g., by macrophages) or otherwise enhance transport of the immunomodulatory nucleic acid molecules to a treatment site for action. Absorption promoters, detergents and chemical irritants (e.g., keratinolytic agents) can enhance transmission of an immunomodulatory nucleic acid molecule composition into a target tissue (e.g., through the skin). For general principles regarding absorption promoters and detergents which have been used with success in mucosal delivery of organic and peptide-based drugs, see, e.g., Chien, Novel Drug Delivery Systems, Ch. 4 (Marcel Dekker, 1992). Examples of suitable nasal absorption promoters in particular are set forth at Chien, supra at Ch. 5, Tables 2 and 3; milder agents are preferred. Suitable agents for use in the method of this invention for mucosal/nasal delivery are also described in Chang, et al., Nasal Drug Delivery, “Treatise on Controlled Drug Delivery”, Ch. 9 and Tables 3-4B thereof, (Marcel Dekker, 1992). Suitable agents which are known to enhance absorption of drugs through skin are described in Sloan, Use of Solubility Parameters from Regular Solution Theory to Describe Partitioning-Driven Processes, Ch. 5, “Prodrugs: Topical and Ocular Drug Delivery” (Marcel Dekker, 1992), and at places elsewhere in the text. All of these references are incorporated herein for the sole purpose of illustrating the level of knowledge and skill in the art concerning drug delivery techniques.

A colloidal dispersion system may be used for targeted delivery of immunomodulatory nucleic acid molecules to specific tissue. Colloidal dispersion systems include macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA and DNA can be encapsulated within the aqueous interior and be delivered to cells in a biologically active form (Fraley, et al., (1981) Trends Biochem. Sci., 6:77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

Where desired, targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various well known linking groups can be used for joining the lipid chains to the targeting ligand (see, e.g., Yanagawa, et al., (1988) Nuc. Acids Symp. Ser., 19:189; Grabarek, et al., (1990) Anal. Biochem., 185:131; Staros, et al., (1986) Anal. Biochem. 156:220 and Boujrad, et al., (1993) Proc. Natl. Acad. Sci. USA, 90:5728). Targeted delivery of immunomodulatory nucleic acid molecules can also be achieved by conjugation of the ISS to the surface of viral and non-viral recombinant expression vectors, to an antigen or other ligand, to a monoclonal antibody or to any molecule which has the desired binding specificity.

Kits for Use in Practiciny the Methods of the Invention

For use in the methods described above, kits are also provided by the invention. Such kits may include any or all of the following: immunomodulatory nucleic acid molecule (with or without antigen, which may be conjugated or unconjugated with the immunomodulatory nucleic acid); a pharmaceutically acceptable carrier (may be pre-mixed with the immunomodulatory nucleic acid molecule) or suspension base for reconstituting lyophilized immunomodulatory nucleic acid molecule; additional medicaments; a sterile vial for each immunomodulatory nucleic acid molecule and additional medicament, or a single vial for mixtures thereof; devices) for use in delivering immunomodulatory nucleic acid molecule to a host; assay reagents for detecting indicia that the anti-inflammatory and/or immunostimulatory effects sought have been achieved in treated animals and a suitable assay device.

In one embodiment, the immunomodulatory nucleic acid, either with or without antigen, is provided in a formulation suitable for administration as a nasal spray or oral spray. The immunomodulatory nucleic acid nasal spray can be provided in a sterile bottle with a nozzle adapted for delivery of the immunomodulatory nucleic acid formulation, e.g., suitable delivery device, such as an inhalation device, and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. All abbreviations and terms used in the examples have their expected and ordinary meaning unless otherwise specified.

### Example I

Murine Model for the Airway Hyperreactivity of Allergic Asthma

Sensitizing-antigen challenged mice of different strains model the airway hyperreactivity seen in allergic asthma. Suitable murine strains for use in modeling the disease include Balb/c mice (which are biased genetically toward the Th2 phenotype and produce enhanced concentrations of IL-4 and IL-5 in response to antigen challenge to CD4+ lymphocytes), C57BL/6 mice (which are IL-5 deficient, for detailed study of IL-5 induced tissue damage in asthma) and W1W′ mice (which are mast cell deficient, for detailed study of mast cell activation in asthma).

Disease modeling mice are conveniently prepared by intraperitoneal or subcutaneous injection of a model sensitizing antigen, ovalbumin (“OVA”) in carrier (e.g., sterile saline or a carrier with adjuvant, such as alum), followed by antigen challenge with aerosolized antigen. For example, mice may be immunized with 25 μg OVA by subcutaneous injection (with or without adjuvant) weekly for 4-6 weeks, then challenged with 2 or 3 weekly aerosolizations of OVA at a concentration of 50 mg/ml in phosphate buffered saline (PBS) delivered in 20 minute intervals or at a concentration of 10 mg/ml 0.9% saline daily for about a week (in three 30 minute intervals daily). Nebulizer devices for use in the aerosolization are available from Aerotech II, CIS-US, Bedford, Mass., with a nasal chamber adapted for murine nasal passages (e.g., a nose-only chamber from Intox Products, Albuquerque, N.Mex.). When driven by compressed air at a rate of 10 liters/min., the devices described produce aerosol particles having a median aerodynamic diameter of 1.4 μm.

Control mice are preferably littermates which are protein-antigen challenged without prior immunization. For further details concerning this animal model, those of skill in the art may wish to refer to Foster, et al., J. Exp. Med., 195-201, 1995; and, Corry, et al., J. Exp. Med., 109-117, 1996.

### Example II

Reduction of Eosinophil Accumulation in Lung Tissue in a Murine Asthma Model by Administration of ISS-ODN

BALB/c mice, 6-10 weeks of age, were prepared as models of allergic asthma as described in Example I (subcutaneous injection of OVA followed by antigen challenge at a concentration of 50 mg OVA/ml PBS). Prior to each inhalation with OVA according to this scheme, sets of 8 mice each were treated as described in Table 1, below. Control mice were antigen challenged but untreated and naive mice were not challenged with antigen. All ISS doses were 100 μg per administration. Dexamethasone (a conventional steroidal anti-inflammatory used in the treatment of asthma) doses were 5 mg/kg/mouse. Priming doses of antigen were 25 μg OVA adsorbed to alum in 0.2 ml phosphate buffered saline (PBS). Challenge doses of antigen were 10 ml of 50 mg OVA/ml PBS. IN=intranasal; IP=intraperitoneal; SC=subcutaneous and N/A=not applicable.

TABLE 1

| Set # | Material Received | Route and Timing |
|---|---|---|
| 1 | Naive mice (no antigen) | N/A |
| 2 | ISS-1 | IN, 1 day before the first |
| | | inhalation |
| 3 | ISS-1 | IN, 1 day before the second |
| | | inhalation |
| 4 | ISS-1 | IN, with the second inhalation |
| 5 | ISS-1 | IN, 2 days after the second |
| | | inhalation |
| 6 | ISS-1 | IP, 1 day before the first |
| | | inhalation |
| 7 | ISS-1 | IP, 1 day before the second |
| | | inhalation |
| 8 | ISS-1 | IP, with the second inhalation |
| 9 | ISS-1 | IP, 2 days after the second |
| | | inhalation |
| 10 | ISS-1 | IT, 2 days after the second |
| | | inhalation |
| 11 | M-ISS | IN, 2 days after inhalaiton |
| 12 | M-ISS | IP, 2 days after the second |
| | | inhalaiton |
| 13 | M-ISS | IT, 2 days after the second |
| | | inhalation |
| 14 | dexamethansone | SC, 2 days after the second |
| | | inhalation |
| 15 | dexamethansone | SC, 7 days after the second |
| | | inhalation |
| 16 | control mice (antigen only) | N/A |

ISS-1 has the nucleotide sequence: 5′-TGACTGTGAACGTTCGAGATGA-3′ (SEQ ID NO. 1) with a phosphothioate backbone and the control M-ISS has the nucleotide sequence: 5′-TGACTGAAGGTTGGAGATGA-3′ (SEQ ID NO. 3) with a phosphothioate backbone.

On day 32, each mouse was bled by tail snip (approximately 50 μl volume) into a 0.1 mM solution of PBS and EDTA. Red blood cells in solution were lysed with 150 mM NH₄Cl and 10 mM KHCO₃ in dH₂O then stained (Wright-Giesma stain). Lung lavage from each mouse was obtained after sacrifice by canalization of the trachea and lavage with 800 microliters PBS, then the lavage product was stained. Bone marrow samples from each mouse were obtained by flushing of extracted femur marrow with PBS. Histological specimens of lung and trachea tissue were obtained from the right lower lobe of the lung and trachea. Specimens were frozen, sectioned to a 5 micron width and stained with DAB peroxidase.

Results are expressed in the Table below as percent eosinophils compared to total leukocytes (inflammatory infiltrate) in each sample, except for the “lung” results, which represent the number ofeosinophils per microscopic field (5 randomly selected fields were evaluated for each sample). In summary, the control mice had an average of 67% eosinophils in the lung/trachea tissue samples, while mice who received the mutant ISS-ODN (M-ISS-ODN) had 52% and 88% (±12%) average accumulation of eosinophils in lung tissue after IP and IN administration, respectively. The higher values for the mice treated with M-ISS-ODN after antigen challenge is most likely owing to the immunoinhibitory properties of M-ISS. See, e.g., U.S. Pat. No. 6,225,292. Mouse sets 7 and 8 therefore model a partially immune incompetent host with allergic asthma.

In startling contrast, the mice pre-treated with the ISS-1 ISS-ODN delivered intranasally had less than about 10% eosinophil accumulation in the lung and trachea when treated after antigen challenge and only about 19% eosinophil accumulation when treated before antigen challenge. These values represent up to an 80% reduction in eosinophil accumulation compared to the control mice and more than a 90% reduction in comparison to M-ISS-ODN (IN) treated mice.

The IP ISS-ODN treated mice fared even better, with a 6% eosinophil accumulation in the lung and trachea on treatment before and after antigen challenge. This value represents an 86% reduction in eosinophil accumulation as compared to the control mice and a 91% reduction as compared to M-ISS-ODN (IP) treated mice.

These data, shown in Table 2, indicate that the IL-5 stimulated eosinophil accumulation in lung tissue which characterizes the late phase of allergic asthma is inhibited by the ISS-ODN therapeutic methods of the invention.

TABLE 2

| | Bone | Broncheoalveolar | | Lung and Tracheal |
|---|---|---|---|---|
| Set # | Marrow | Lavage | Blood | Tissue |
| 1 (naive) | 3 ± 2 | 0 | 2 ± 1 | 2 ± 1 |
| 2 (ISS) | 5 ± 1 | 10 ± 2 | 3 ± 1 | 8 ± 1 |
| 3 (ISS) | 12 ± 1 | 17 ± 4 | 6 ± 2 | 19 ± 5 |
| 4 (ISS) | 5 ± 1 | 3 ± 1 | 2 ± 1 | 6 ± 1 |
| 5 (ISS) | 8 ± 2 | 4 ± 3 | 3 ± 1 | 6 ± 4 |
| 6 (ISS) | 10 ± 1 | 10 ± 1 | 4 ± 1 | 16 ± 4 |
| 7 (M-ISS) | 13 ± 1 | 51 ± 3 | 10 ± 1 | 88 ± 12 |
| 8 (M-S) | 13 ± 1 | 43 ± 3 | 10 ± 1 | 52 ± 14 |
| 9 (control) | 3 ± 2 | 42 ± 4 | 14 ± 3 | 67 ± 5 |

### Example III

Antigen Independent Reduction of Eosinophil Accumulation in Lung Tissue

To evaluate whether the eosinophil suppression demonstrated by the data in Example II is dependent upon immune stimulation by the ISS-ODN, mice were sensitized to OVA using a conventional, Th2 stimulatory adjuvant (alum), treated with ISS-ODN or a control, and measured for eosinophil suppression before ISS-ODN stimulation of the mouse immune system would be expected to occur.

More specifically, groups of four mice were immunized with 25 μg OVA in 1 mg alum by subcutaneous injection on days 1, 7, 14 and 21. This immunization protocol is known to stimulate a Th2 type response to the antigen in mice. On day 27, one group of animals received 100 μg of the ISS-1 ISS-ODN (SEQ ID NO:1) described in Example I by intraperitoneal administration. A control group received the mutant M-ISS-ODN described in Example I by the same route.

On day 28, the animals in each group received 10 mg OVA/ml phosphate buffered saline by inhalation for 30 minutes. On day 30, some of the animals in each group received a second injection of ISS-ODN or M-ISS-ODN and the animals who had not been treated on day 27 were treated with ISS-ODN or MISS-ODN. The inhalation challenge with OVA was repeated on day 31 and the animals were sacrificed for eosinophil counting within 24 hours.

The results of this experiment are set forth in Table 3 below. Animals that received two treatments with ISS-ODN on days 27 and 30 had only 5.8% eosinophils in the broncheo-alevolar fluid (BALF) lavage on day 32, even though immune stimulation by the ISS-ODN would be minimal so shortly after treatment. Even after only one treatment with ISS-ODN (on day 30),eosinophil accumulation in the BALF the treated animals was limited to 10.3%. In contrast, the control animals twice treated with MISS-ODN had 42.3% eosinophils in extracted BALF.

TABLE 3

| | Treated on | | | |
|---|---|---|---|---|
| Animals | Day 28 | Blood | Bone Marrow | BALF |
| ISS-ODN | Yes | 1.9% ± 0.8 | 5.8% ± 2.5 | 5.8% ± 2.8 |
| M-ISS-ODN | Yes | 9.8% ± 2.1 | 13.0% ± 0.9 | 42.3% ± 3.5 |
| ISS-ODN | No | 3.5% ± 0.6 | 10.5% ± 1.4 | 10.3% ± 1.3 |

These data establish that practice of the invention can inhibit allergic inflammation in animals and that the inhibition can occur as quickly as one day after treatment.

### Example IV

Selective Induction of a Th1 Response in a Host after Administration of an ISS Containing Plasmid

In mice, IgG 2A antibodies are serological markers for a Th1 type immune response, whereas IgG 1 antibodies are indicative of a Th2 type immune response. Th2 responses include the allergy associated IgE antibody class; soluble protein antigens tend to stimulate relatively strong Th2 responses. In contrast, Th1 responses are induced by antigen binding to macrophages and dendritic cells.

To determine which response, if any, would be produced by mice that received ISS-ODN according to the invention, nine groups of Balb/c mice were immunized with 10 μg β-galactosidase protein (conjugated to avidin; Sigma, St. Louis, Mo.) to produce a model allergic phenotype and treated as shown in Table 4, below:

TABLE 4

| Mouse Group | ISS-ODN Treatment |
|---|---|
| 1 | None (β-gal) |
| 2 | ISS-1 (ISS-ODN) injected with the antigen |
| 3 | M-ISS injected 72 hours. after the antigen (same site) |
| 4 | M-ISS (M-ISS-ODN) injected with the antigen |
| 5 | M-ISS injected 72 hours after the antigen (same site) |

At 2 week intervals, any IgG 2a and IgG 1 to (β-galactosidase present in the serum of each mouse were measured by enzyme-linked immunoabsorbent assay (using antibodies specific for the IgG 1 and IgG 2A subclasses) on microtiter plates coated with the enzyme.

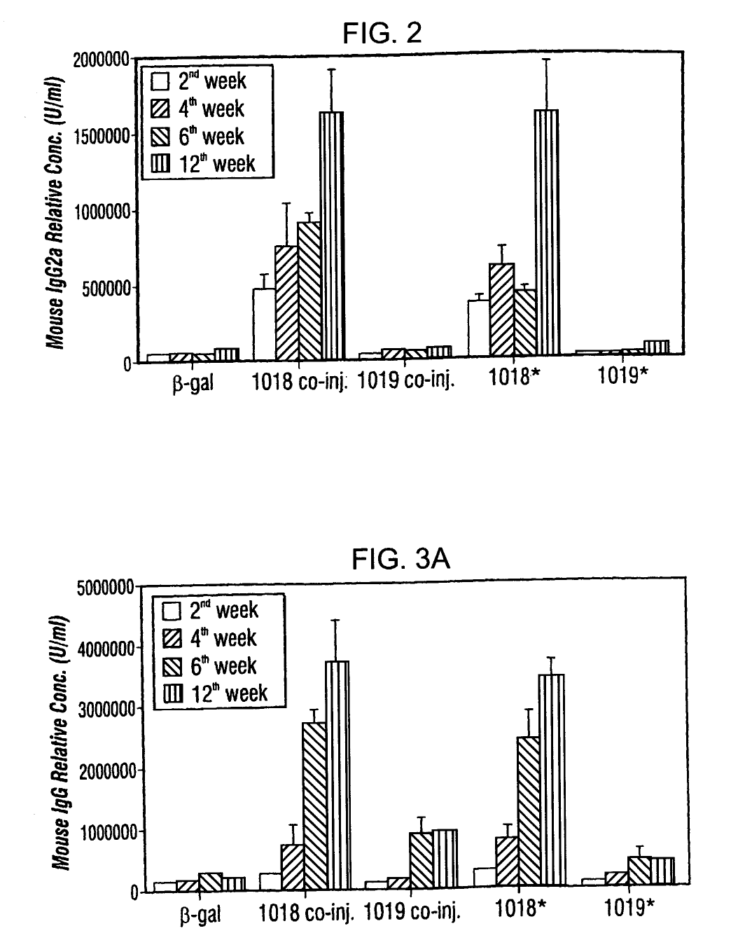

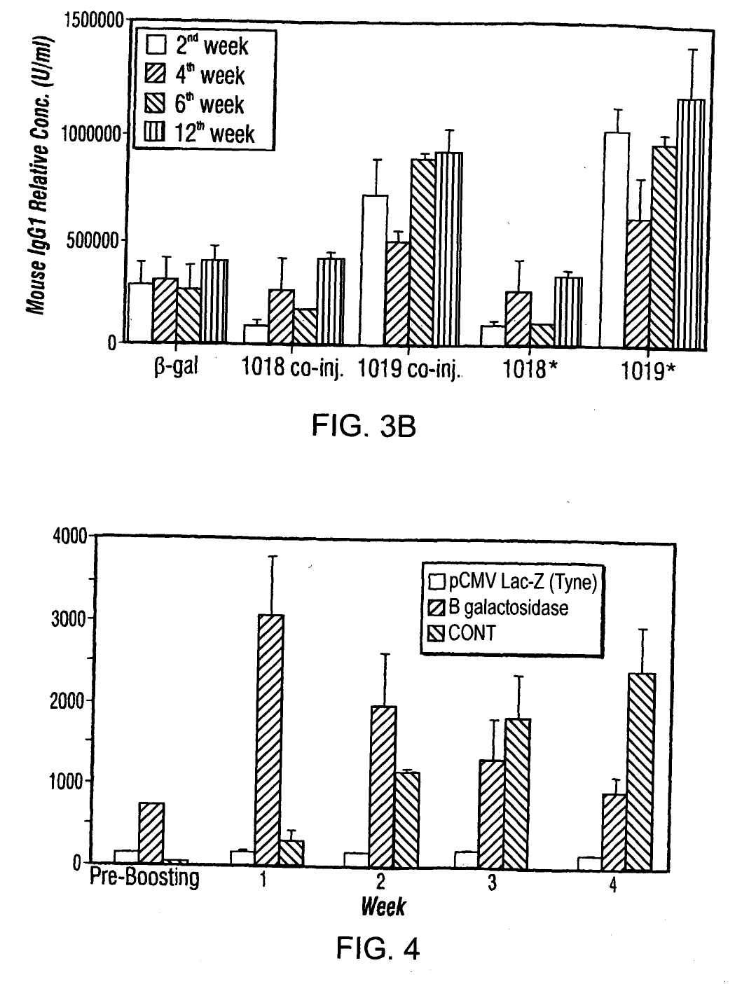

As shown in FIG. 2, only the mice that received the ISS-ODN produced high titers of IgG 2A antibodies, which increased in number over a period of 12 weeks. As shown in FIG. 3, immunization of the mice with the antigen itself or with the mutant ISS-ODN induced production of relatively high titers of IgG 1 antibodies. The data shown in FIGS. 2 and 3 comprise averages of the values obtained from each group of mice.

These data indicate that a selective Th1 response is induced by administration of an ISS-ODN according to the invention to an antigen-challenged host. Further, the data indicate that ISS-ODN administration according to the invention biases the immune system toward the Th1 phenotype on antigen challenge, even when the ISS-ODN are administered before antigen challenge (in this instance, 72 hours before challenge).

### Example V

Suppression of IgE Antibody Response to Antigen by Immunization with Antigen-Encoding Polynucleotides

To demonstrate the IgE suppression achieved through stimulation of a Th1 type cellular immune response in preference to a Th2 type cellular immune response, five to eight week old Balb/c mice were immunized with one of two recombinant expression vectors: ISS-ODN containing pCMV-LacZ (which contains two copies of nucleotide sequences similar to the ISS-1 ISS-ODN) or a control plasmid, pCMV-BL. A third group of the mice received injections of antigen (β-galactosidase). Plasmid DNA was purified and its endotoxin content reduced to 0.5-5 ng/1 mg DNA by extraction with TRITON X-114 (Sigma, St. Louis, Mo.). Before inoculation, pDNA was precipitated in ethanol, washed with 70% ethanol and dissolved in pyrogen free normal saline.

Immunization was by intradermal injection of plasmid DNA loaded onto separate tynes of a MONOVACC® multiple tyne device (Connaught Lab, Inc., Swiftwater, Pa.). Briefly, the tyne devices were prepared after extensive washing in DDW and overnight soaking in 0.5% SDS (sulfated dodecyl saline), washed again in DDW, soaked overnight in 0.1N NaOH, washed again in DDW and dried at 37° C. for 8 hours. Six μl of plasmid DNA dissolved in normal saline were pipetted onto the tynes of the tyne device just prior to each inoculation described below. The total amount of pDNA loaded on the device per inoculation was 25 μg each of pCMV-Lac-Z and pCMV-BL. For purposes of estimating actual doses, it was assumed that less than 10% of the pDNA solution loaded onto the tyne device was actually introduced on injection of the tynes into intradermal tissue.

Each mouse was treated 3 times with 2 inoculations of each plasmid in a one-week interval injected intradermally at the base of the tail. Another group of mice received a single intradermal injection in the base of the tail of 10 μg of galactosidase protein (dissolved in 50 μl of normal saline) in lieu of pDNA.

Toward inducing an IgE antibody response to subsequent sensitizing-antigen challenge, each group of mice was injected once intraperitoneally with 0.1 ml of phosphate buffered saline (PBS) solution containing 1 μg of antigen (galactosidase; Calbiochem, San Diego, Calif.) and 3 mg of ALUM aluminum hydroxide as adjuvant (Pierce Chemical, Rockford, Ill.) 14 weeks after the initial immunization. Total IgE was assayed in sera from the mice 4 times over the subsequent 4 consecutive weeks.

IgE was detected using a solid phase radioimmunoassay (RAST) in a 96 well polyvinyl plate (a radioisotopic modification of the ELISA procedure described in Coligan, “Current Protocols In Immunology”, Unit 7.12.4, Vol. 1, Wiley & Sons, 1994), except that purified polyclonal goat antibodies specific for mouse E chains were used in lieu of antibodies specific for human Fab. To detect antiLac-Z IgE, the plates were coated with β-galactosidase (10 μg/ml). The lowest IgE concentration measurable by the assay employed was 0.4 ng of IgE/ml.

Measuring specifically the anti-antigen response by each group of mice, as shown in FIG. 4, anti-Lac-Z IgE levels in the ISS-ODN containing plasmid injected mice were consistently low both before and after boosting (averaging about 250 CPM in RAST), while the protein injected mice developed high levels of anti-Lac-Z, particularly after the first antigen booster injection, when anti-Lac-Z levels in the mice rose to an average of about 3000 CPM. Consistent with acquisition of tolerance, anti-Lac-Z IgE levels in the protein injected mice declined over time, but continued to rise in the control mice that had not received any immunization to β-galactosidase.

These data show that the ISS-ODN containing plasmid injected mice developed an antigen specific Th1 response to the plasmid expression product with concomitant suppression of IgE production, while tolerance was acquired in the protein injected mice only after development of substantially higher levels of antigen specific IgE antibodies.

### Example VI

IL-4, IL-5, IL-10 AND IFN-γ Levels and CD4+ Lymphocyte Proliferation, in Mice after Delivery of ISS

BALB/c mice were injected intravenously with 100 μg of ISS-1, M-ISS or a random sequence control (DY1043) then sacrificed 24 hrs later. Splenocytes were harvested from each mouse.

96 well microtiter plates were coated with anti-CD3 antibody (Pharmingen, La Jolla, Calif.) at a concentration of 1 μg/ml of saline. The anti-CD3 antibody stimulates T cells by delivering a chemical signal which mimics the effects of binding to the T cell receptor (TCR) complex. The plates were washed and splenocytes added to each well (4×10⁵/well) in a medium of RPMI 1640 with 10% fetal calf serum. Supernatants were obtained at days 1, 2 and 3.

Th2 cytokine (IL-4, IL-5 and IL-10) levels were assayed in the supernatants using a commercial kit; Th1 cytokine (IFN-γ) levels were assayed with an anti-IFN-γ murine antibody assay (see, e.g., Coligan, “Current Protocols in Immunology”, Unit 6.9.5., Vol. 1, Wiley & Sons, 1994). Relatively high levels of IL-4 and IL-10 with low levels of INF- would be expected in mice with a Th2 phenotype, while relatively low levels of IL-4 and IL-10 with high levels of INF-γ would be expected in mice with a Th1 phenotype. Relatively high levels of IL-5 characterize a proinflammatory milieu, while the converse is true of relatively low levels of IL-5.

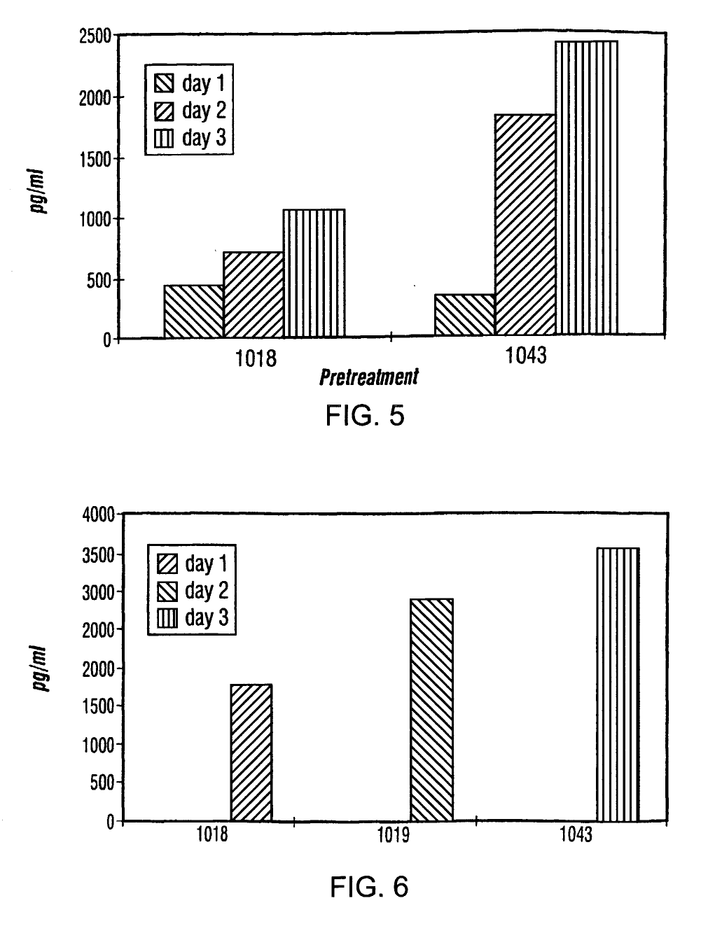

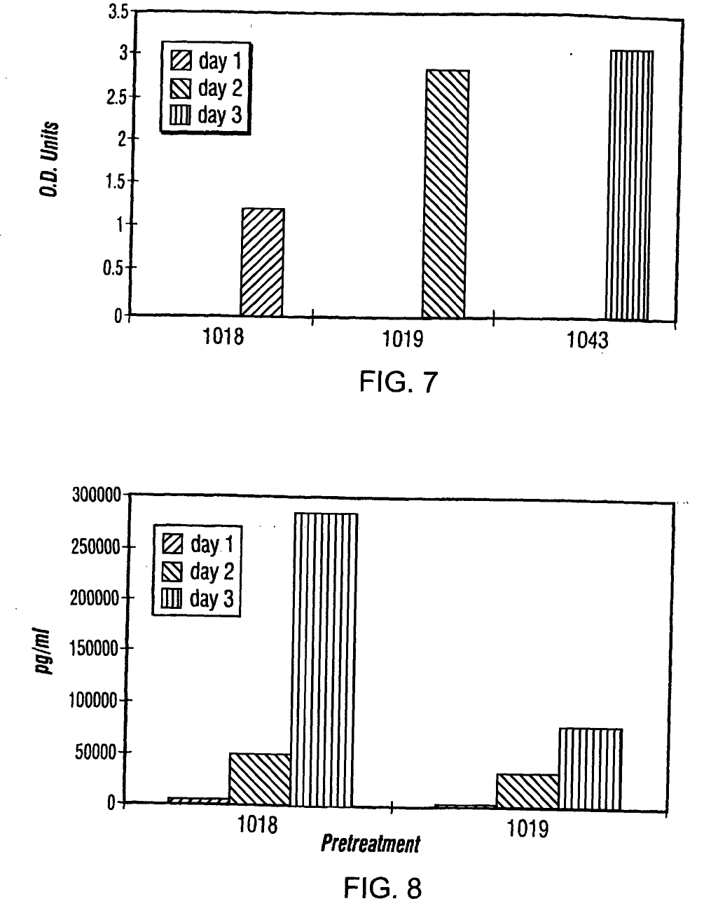

As shown in FIGS. 5 and 6, levels of anti-CD3 stimulated IL-4 and IL-10 secretion in ISS-1 treated mice were substantially lower than in the control mice. Levels in the M-ISS mice were intermediate. Levels of pro-inflammatory IL-5 were reduced in ISS-1 treated mice to a comparable extent (FIG. 7).

Levels of T cell proliferation in response to antigen challenge were greatly reduced in ISS-1 (ISS-ODN) treated mice as compared to M-ISS (mutant ISS-ODN) treated and control mice. This suppression of T cell proliferation was reversible on adminstration of IL-2, demonstrating that the suppression was due to Th2 anergy in the ISS-ODN treated mice (see, Table 5 below).

TABLE 5

| Treatment | Control (CPM) | ISS-ODN (CPM) | M-ODN (CPM) |
|---|---|---|---|
| OVA (50 μg/ml) | 40680 ± 5495 | 15901 ± 4324 | 42187 ± 13012 |
| OVA + IL-2 | 65654 ± 17681 | 42687 ± 6329 | 79546 ± 10016 |
| (1.5 ng/ml) | | | |
| OVA-IL-2 | 60805 ± 19181 | 57002 ± 10658 | 60293 ± 5442 |
| (15 ng/ml) | | | |

Levels of Th1 stimulated IFN-γ secretion were greatly increased in the ISS-1 treated mice, but substantially reduced in the M-ISS treated mice (as compared to the control), indicating stimulation of a Th2-type milieu in the latter mice (FIG. 8). Additional data demonstrating these results are shown in the Table below. “b/f” in the Table refers to before; “1st”, “2nd” and “each” refer to administration of the compound before the 1st or 2nd antigen challenge.

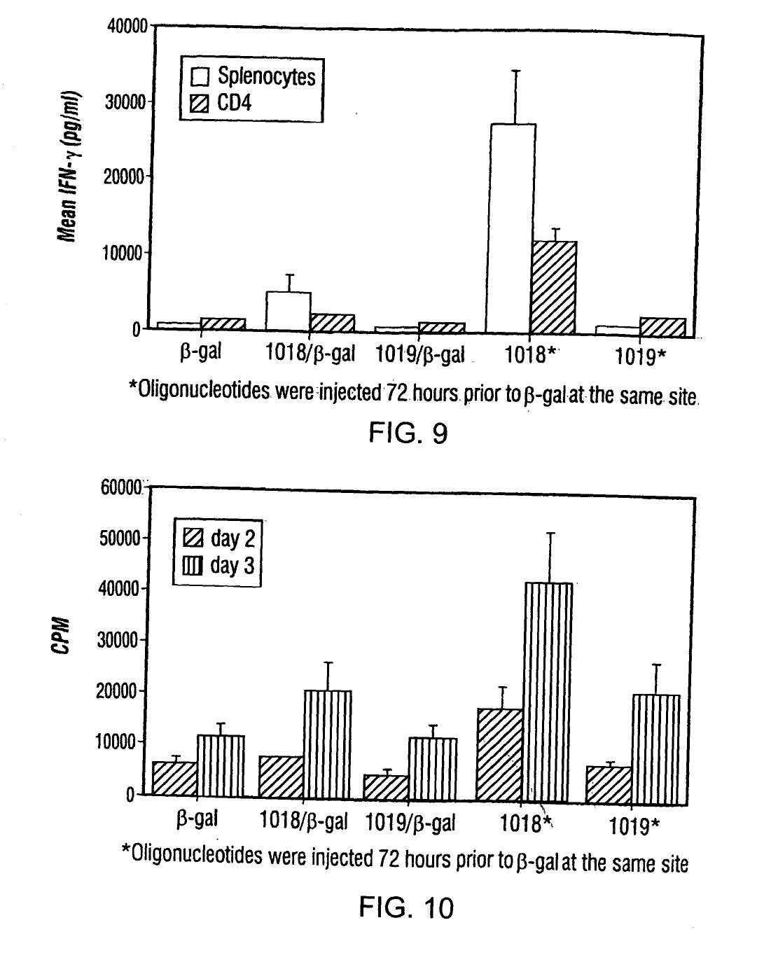

Importantly, treatment of mice before antigen challenge is even more effective in shifting the immune response on antigen challenge to a Th1 phenotype than is post-challenge treatment. As shown in FIGS. 9 and 10, antigen primed (but unchallenged) animals injected with ISS-ODN M-ISS 72 hours before antigen challenge (with β-galactosidase) mounted a more robust Th1-type immune response to the antigen than did their post-challenge treated littermates or littermates treated pre-challenge with a mutant, inactive oligonucleotide (M-ISS), as measured by increased IFNγ- secretion (FIG. 9) and CD4⁺ lymphocyte proliferation (FIG. 10). The data are summarized in Table 6, below.

TABLE 6

| Set # | IL-5 (pg/ml) | IFN_{γ.}-(pg/ml) |
|---|---|---|
| 1 (naive) | .>20 | .>20 |
| 2 (ISS-1) in b/f 1st | 466 ± 40 | 246 ± 86 |
| 3 (ISS-1) b/f 2nd | 531 ± 109 | 168 ± 22 |
| 4 (ISS-1) in with 2nd | 575 ± 90 | 98 ± 44 |
| 5 (ISS-1) in b/f each | 200 ± 66 | 443 ± 128 |
| 6 (ISS-1) ip; b/f 1st | 190 ± 52 | 664 ± 61 |
| 7 (ISS-1) ip; b/f 2nd | 421 ± 102 | 252 ± 24 |
| 8 (ISS-1) ip; with 2nd | 629 ± 110 | 104 ± 15 |
| 9 (ISS-1) ip; b/f each | 121 ± 18 | 730 ± 99 |
| 10 (ISS-1) it; b/f each | 191 ± 49 | 610 ± 108 |
| 11 (M-ISS) in; b/f each | 795 ± 138 | 31 ± 22 |
| 12 (M-ISS) it; b/f each | 820 ± 122 | 33 ± 33 |
| 13 (M-ISS) it; b/f each | 657 ± 52 | 102 ± 57 |
| 14 (steroid) sc; b/f each | 424 ± 90 | .>20 |
| 15 (steroid) sc; daily | 252 ± 96 | .>20 |
| 16 (control) not treated | 750 ± 124 | 24 ± 21 |

Further, ISS administered according to the invention suppress Th2 cytokine release from Th2 sensitized mouse cells (splenocytes harvested from OVA-primed mice, then incubated for 72 hours with 100 μg/ml OVA in vitro). IS SODN treatment took place either 1 (−1) or 3 (−3) days before sacrifice. These data are shown in Table 7 below:

TABLE 7

| Group | IL-3 (pg/ml) | IL-5 (pg/ml) | IFN-γ (pg/ml) |
|---|---|---|---|
| Control | 1299 ± 89 | 657 ± 52 | .>20 |
| ISS-ODN (−1) | 309 ± 26 | 112 ± 18 | .>20 |
| ISS-ODN (−3) | 463 ± 48 | 144 ± 27 | .>20 |
| ISS-ODN (−1) | 964 ± 81 | 508 ± 77 | .>20 |

### Example VII

Expression of a Viral Protein Following Intradermal Injection of a Naked Gene Expression Vector

To demonstrate the competence of naked gene expression vectors of the invention for expression in the dermis, the gene for influenza ribonucleoprotein (NP) was subcloned into a pCMV plasmid. NP genes from numerous strains of influenza are known in the art and are highly conserved in sequence among various strains (see, e.g. Gorman, et al., J. Virol, 65:3704, 1991).

Four eight-week-old Balb/c mice were injected three times with 15 μg of pCMV-RNP suspended in 100 μl of HBSS. Injections were made intradermally at the base of the tails at two week intervals. CTLs recognize antigens presented by class I MHC molecules and play an important role in the elimination of virally infected cells. Intramuscular (i.m.) immunization by means of cDNA expression vectors should be an effective method to introduce antigen into class I MHC molecules and thus stimulate CTL responses. In this study, intradermal (i.d.) injection of a plasmid containing the influenza nucleoprotein (NP) antigen gene induced both NP-specific CTL and high titers of anti-NP antibodies. These antibodies reached a maximum 6 weeks after injection and persisted unchanged for at least 28 weeks, in the absence of local inflammation.

Plasmid DNA was purified by CsCl banding in the presence of ethidium bromide and was stored frozen in 10 mM Tris-HCl, 0.1 mM EDTA, pH 8.0. Before injection, the plasmid was precipitated in ethanol and dissolved in normal saline containing 0.1 mM EDTA.

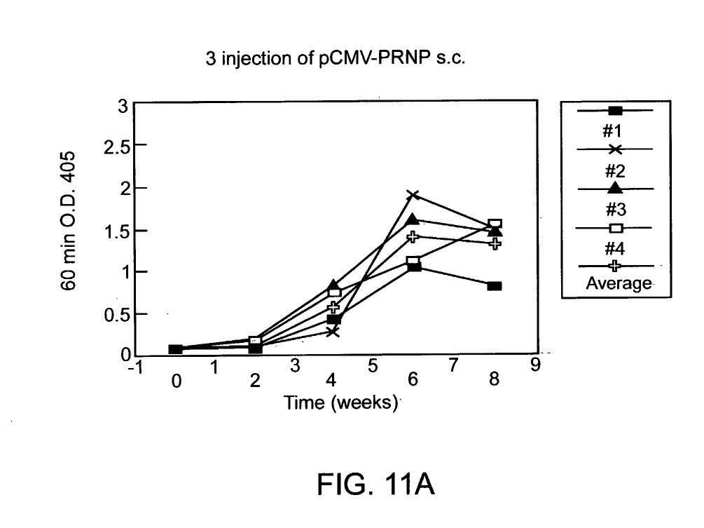

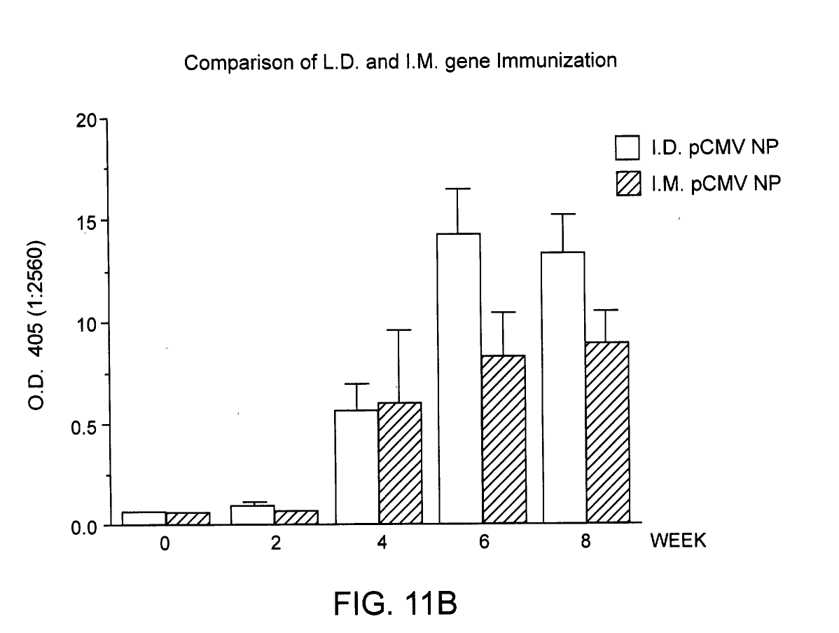

The presence of anti-NP IgG in serum was measured by ELISA substantially as described in Viera, et al., Int. Immunl., 2:487, (1990). The results of this assay are shown in FIG. 11a; all of the animals developed high titer anti-NP antibodies, which persisted for more than 20 weeks. As shown in FIG. 11b, the intradermal injections appeared to give about four fold higher antibody titers than intramuscular injections of equivalent amounts of plasmid DNA.

The axes of FIGS. 11a and 11b represent, respectively, the ELISA titer (mean, 1 ounce) against time. Serum dilution for all graph points is 2560.

### Example VIII

In vivo Antibody Responses to the Immunostimulatory Polynucleotides of the Invention

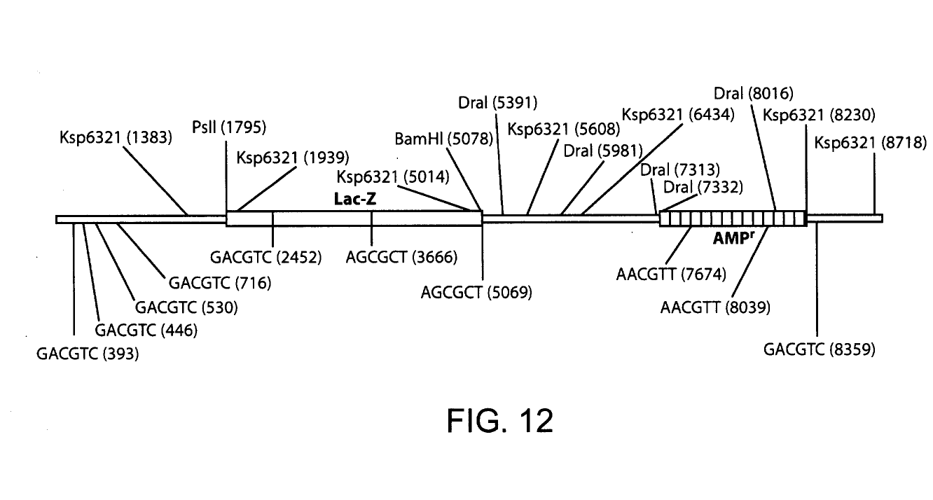

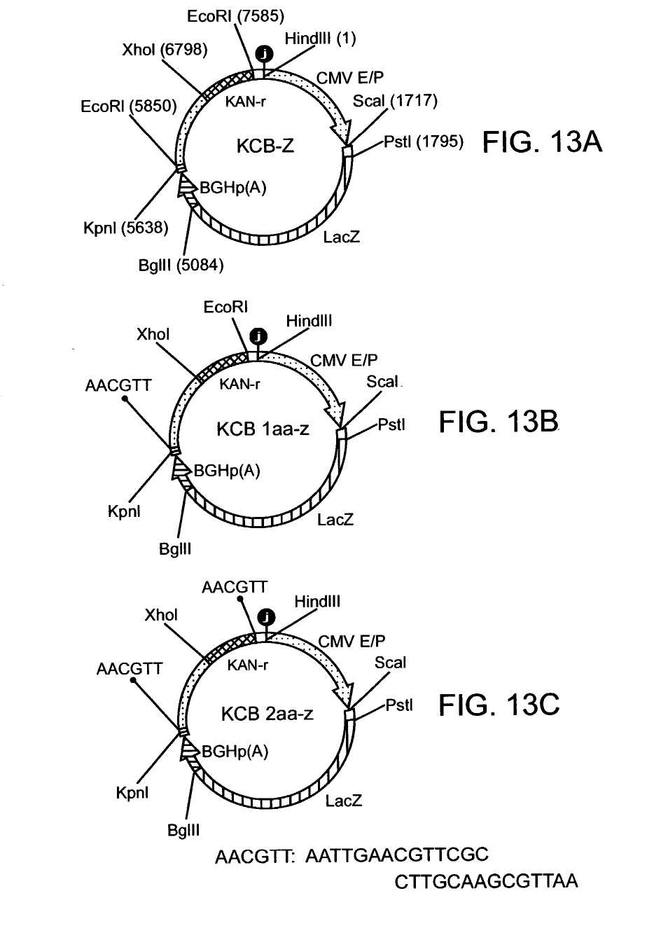

To compare humoral immune responses to naked gene expression vectors containing the immunostimulatory polynucleotides of the invention to humoral immune responses to vectors lacking such polynucleotides, the pCMV-LacZ plasmid shown schematically in FIG. 1 (which includes two copies of the immunostimulatory polynucleotide of SEQ.ID.No.1) was modified to substitute a gene encoding an enzyme which confers kanamycin resistance (KanR). The resulting plasmid (pKCB-LacZ) lacks any of the immunostimulatory polynucleotides of the invention (see, vector maps in FIG. 12 (pCMV-LacZ) and FIGS. 13a-c (pKCB-LacZ)). In contrast, the AmpR containing pCMV-LacZ plasmid includes the AACGTT (SEQ.ID.No.1) palindromic sequence at two separate locations in the vector within the AmpR gene.

Four Balb/c mice per group were each injected intradermally at the base of the tail with 50 μg of either the pCMV-LacZ or pKCB-LacZ plasmids. Each injection was repeated twice at one week intervals. A third group of mice was injected with pKCB-LacZ and supplementally injected with pUC-19, a plasmid which includes the AmpR gene. As a control, a fourth group of mice was injected with a non-specific bacterial DNA. For comparison of the overall immune response elicited, a fifth group was injected with a naked gene expression vector which operatively encodes GM-CSF (granulocyte monocyte colony stimulating factor). Anti-antigen antibody production was measured by serum ELISA after 6 weeks.

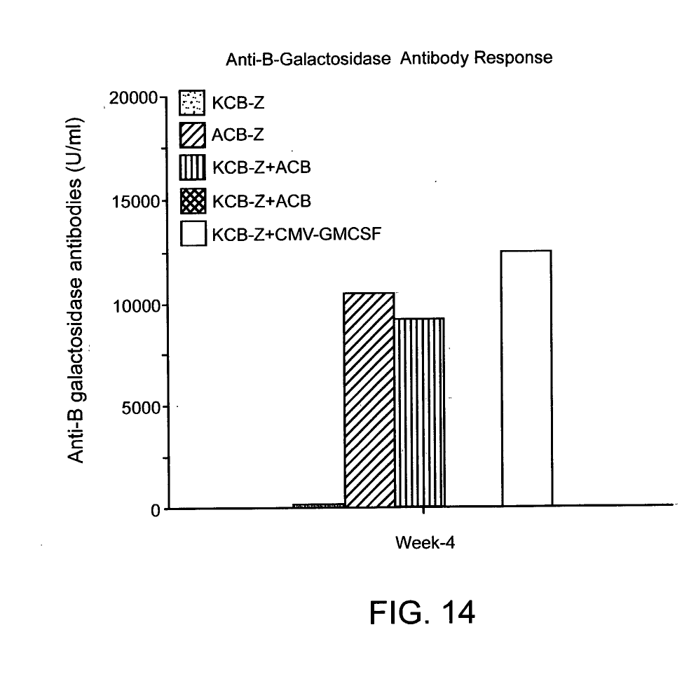

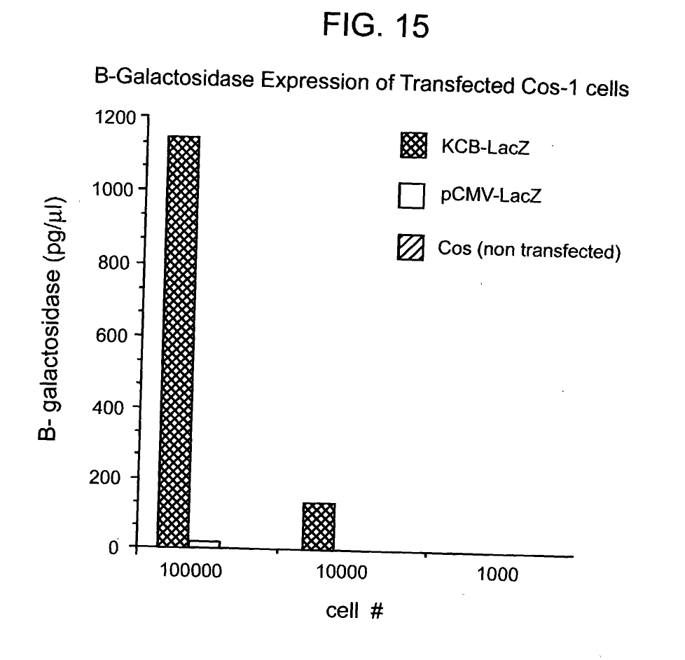

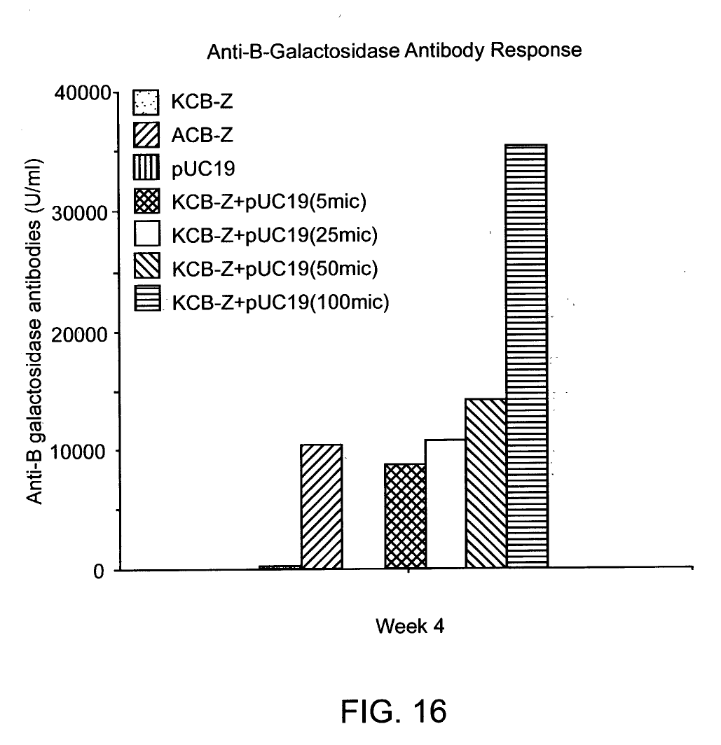

As shown in FIG. 14, the mice injected with pCMV-LacZ produced antibodies against the expressed LacZ reporter molecule. However, no antibody formation was detected in the sera of the mice who received the pKCB-LacZ plasmid, despite the higher level of LacZ expression achieved by the vector (detected as a measure of β-galactosidase activity in Chinese hamster ovary cells transfected separately with each vector; see, FIG. 15. Yet anti-LacZ antibody production was restored with coadministration of pKCB-LacZ and pUC-19 FIG. 16, although no such response was detected after injection of the control plasmid (id.). The enhancing effect of the pUC-19 vector exceeded even the response to the GM-CSF encoding vector (id.).

To determine the effect of the immunostimulatory polynucleotides of the invention on humoral immune responses, the pKCB-LacZ plasmid was modified to include one or two copies of the AACGTT polynucleotide palindrome found in the AmpR gene (pKCB-laaZ (1 copy) and pKCB-2aaZ (2 copies)). For comparison, groups of pKCBLacZ and pCMV-LacZ injected mice were also injected with, respectively, KCB or CMV plasmids which lacked the LacZ reporter molecule. Antibody responses to LacZ were measured at 4 weeks after 3 weeks of immunization as described above.

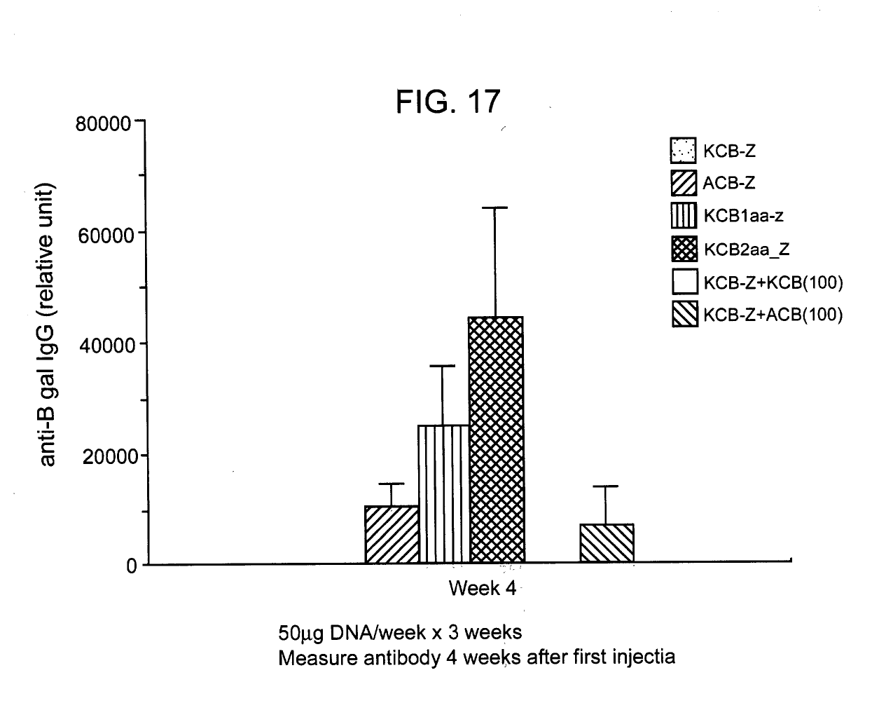

As shown in FIG. 17, virtually no antibody response to LacZ was measured in the mice injected with pKCB-LacZ or pKCB-LacZ/pKCB, while antibody responses were detected in the mice injected with pCMV-LacZ and pCMV-LacZ/pCMV. Moreover, the mice injected with the modified KCB plasmids produced substantially greater antibody titers than even the mice injected with the pCMV plasmids, which responses increased in proportion to the number of copies of the AACGTT polynucleotide (SEQ.ID.No.1) present in the plasmid. The enhanced response as compared to the pCMV plasmids (which contain two copies of the AACGTT polynucleotide) is probably attributable to the greater levels of antigen expression achieved by the KCB vectors (see, FIG. 15).

### Example IX

In vivo CTL Activity in Response to the Immunostimulatory Polynucleotides of the Invention

To determine whether the immunostimulatory polynucleotides of the invention (i.e., palindromic, CG containing sequences) stimulate cellular as well as humoral responses, the lytic activity of CTLs after immunization of mice with either pKCB-LacZ or pCMV-LacZ was tested. A control group of mice was immunized with the antigen in alum.

3 to 6 weeks after immunization (performed as described in Example VIII), the mice were sacrificed and splenocytes were removed for use in standard mixed lymphocyte cultures. The cultures were grown in the presence of a known synthetic β-galactosidase peptide. The cultures were assayed for anti-LacZ CTL activity 5-6 days, measured as a function of the percent lysis of cells exposed to the antigen by pulsing versus the effector (antigen):target ratio.

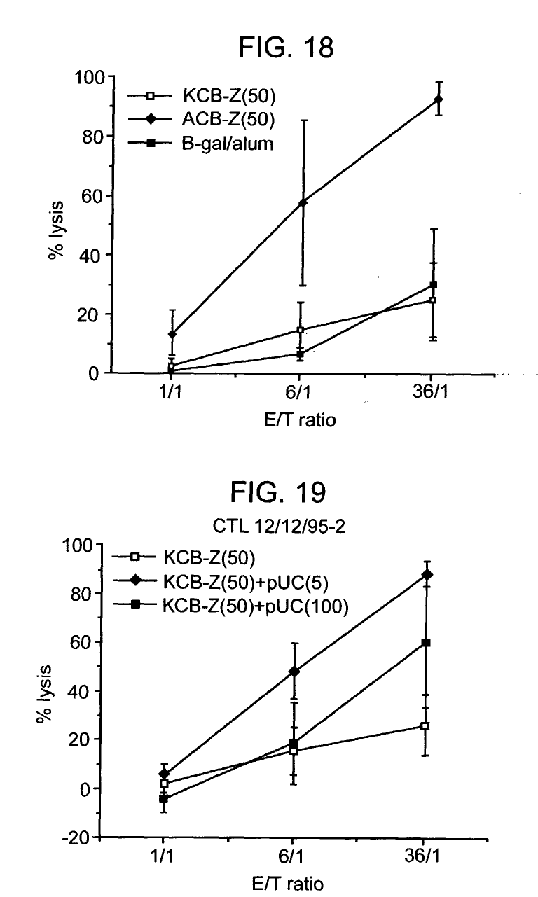

As shown in FIG. 18, as the effector:target ratio was increased, the CTL activity in cultures of cells from the pCMV-LacZ injected mice increased from about 18% to nearly 100%. In contrast, the CTL activity in cultures from the pKCB-LacZ and control injected mice barely exceeded 20% lytic activity even when the effector:target ratio was raised to 36:1.

To determine the effect of the two copies of the immunostimulatory polynucleotide (AACGTT) of SEQ.ID.No.1 in the pCMV-LacZ plasmid, another group of pKCB-LacZ injected mice received a co-injection of either 5 μg or 100 μg of pUC-19. An increase in CTL activity to nearly 60% lysis was achieved in the latter group (FIG. 19).

### Example X

Immune Response to Viral Challenge by Mice Intradermally Injected with Naked Gene Expression Vectors Containing Immunostimulatory Polynucleotides of the Invention

To test whether immunity generated by vaccination with naked gene expression vectors of the invention could protect animals from a lethal viral challenge, groups of 10 Balb/c mice were injected intradermally 3 times with 15 μg of a pCMV plasmid (pCMV-NP) which contained two copies of the immunostimulatory polynucleotide of SEQ.ID.No. 1 and the NP gene from an H1N1 strain of influenza virus (A/PR/8/34; provided by Dr. Inocent N. Mbawvike at the Baylor College of Medicine, U.S.) Control groups included uninfected animals as well as animals injected with an irrelevant plasmid (pnBL3).

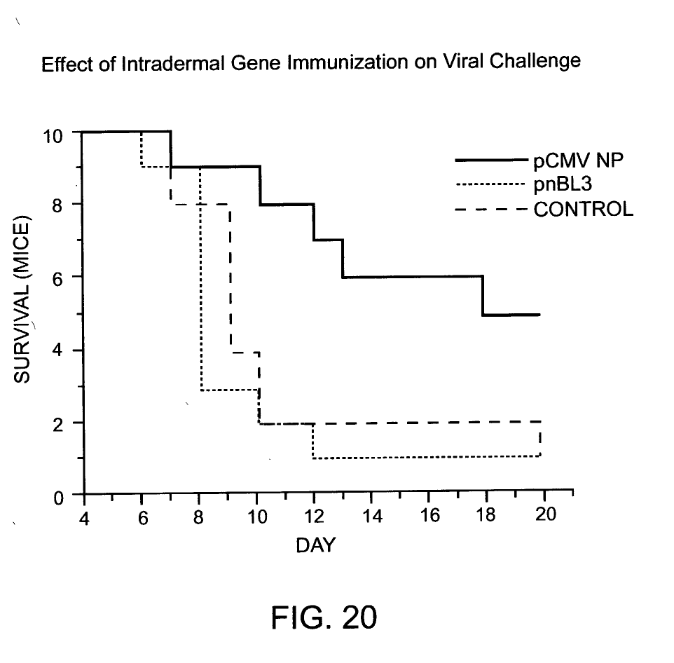

Six weeks after the initial plasmid injections, the animals were challenged with a LD₉₀ dose of an H3N2 influenza strain (A/HK/68); also provided by Dr. Mbawuike). Intradermally vaccinated mice were significantly protected from the challenge (p<0.01) as compared to unvaccinated control mice; see, FIG. 20 (a Kaplan-Meyer survival curve).

### Example XI

Prolonged Immunologic Memory after Intradermal Administration of Naked Polynucleotides Induced by Antigen Stimulation of T Cells

To test whether the protective effect observed in the mice described in Example X included long-term immunologic protective memory, 0.1, 1, 10 and 100 μg of naked gene expression vectors (0.5-5 ng/1 mg DNA endotoxin content) encoding the E. coli enzyme β-galactosidase under the control of the CMV promoter were administered to groups of 4 mice\dosage\route either intramuscularly (“IM”) or intradermally (“ID”). Each plasmid included two copies of the immunostimulatory polynucleotide of SEQ.ID.No.1 (pCMV-LacZ).

As a control, another group of 4 mice\dosage received 100 μg β-galactosidase protein (“PR”) intradermally. All injections were made using 50 μl normal saline as carrier. IM and ID injections were made with a 0.5 ml syringe and a 28.5 gauge needle. Antibodies were thereafter measured by enzyme-linked immunoabsorbent assay at 2-week intervals.

Total anti-β galactosidase antibodies were measured using β-galactosidase (Calbiochem, Calif.) as the solid phase antigen. Microtiter plates (Costar, Cambridge, Mass.) were coated with 5 μg of antigen dissolved in 90 mM borate (pH 8.3) and 89 mM NaCl (i.e., borate buffered saline; BBS) overnight at room temperature and blocked overnight with 10 mg/ml of bovine serum albumin in BBS.

Serum samples were serially diluted in BBS starting at a 1:40 dilution for the first 8 weeks, then a 1:320 dilution thereafter. These samples were added to the plates and stored overnight at room temperature. Plates were washed in BBS+0.05% polysorbate 20, then reacted with a 1:2000 dilution of alkaline phosphatase labeled goat anti-mouse IgG antibody (Jackson Immunoresearch Labs., West Grove, Pa.) for 1 hour at room temperature, or were reacted with a 1:2000 dilution of alkaline phosphatase labeled goat anti-mouse IgG 1 antibody (Southern Biotech of AL), or were reacted with a 1:500 dilution of alkaline phosphatase labeled rat anti-mouse IgG 2A antibody (Pharmingen, of Calif.), under the same conditions. Plates were washed again, then a solution of 1 mg/ml of p-nitrophenol phosphate (Boehringer-Mannheim, Indianapolis, Ind.) in 0.05 M carbonate buffer (pH 9.8), containing 1 mM MgCl₂ was added. Absorbance at 405 nm was read 1 hour after addition of substrate to the plates.

Lesser antibody responses were measured in the animals that had received the pCMV Lac-Z plasmids by IM injection than by ID injection.

To assess for T cell memory, the animals were then boosted with 0.5 μg of PR at a separate site by ID injection. If these animals had developed memory T cells to control production of antibody to β-galactosidase, they would be expected to mount a more vigorous immune response after boosting with soluble protein antigen than had been demonstrated in response to the priming dose of antigen.

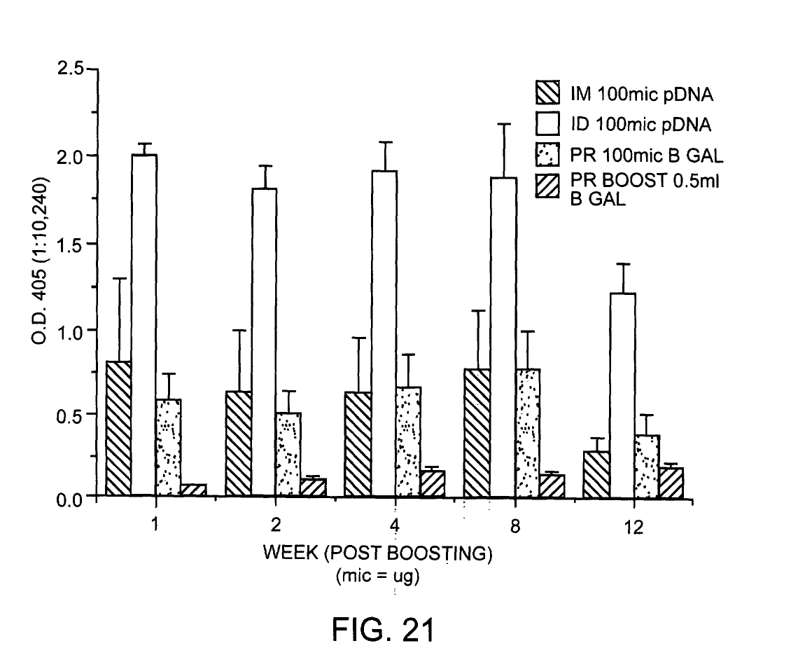

As shown in FIG. 21, it is clear that the animals which had received ID injections of pCMV-LacZ plasmid had developed substantially better immunological memory than did animals which had received either IM injections of plasmid or of PR. Further, the memory which was developed by the ID injected animals persisted for a minimum of about 12 weeks.

### Example XII

Selective Induction of a Th1 Response after Intradermal Administration of Naked Polynucleotides

In mice, IgG 2A antibodies are serological markers for a TH 1 type immune response, whereas IgG 1 antibodies are indicative of a TH2 type immune response. TH2 responses include the allergy-associated IgE antibody class; soluble protein antigens tend to stimulate relatively strong TH2 responses. In contrast, TH1 responses are induced by antigen binding to macrophages and dendritic cells. TH 1 responses are to be of particular importance in the treatment of allergies and AIDS.

To determine which response, if any, would be produced by mice that received naked gene expression vectors according to the invention, mice were vaccinated with the pCMV-LacZ vector described in Example XI or protein as described in Example XI. At 2-week intervals, any IgG 2a and IgG 1 to β-galactosidase were measured by enzyme-linked immunoabsorbent assay (using antibodies specific for the IgG 1 and IgG 2A subclasses) on microtiter plates coated with the enzyme.

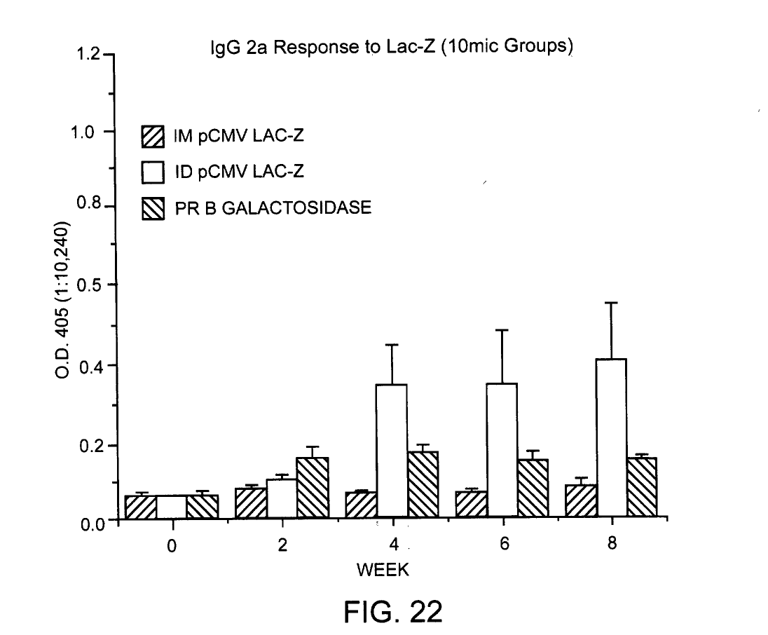

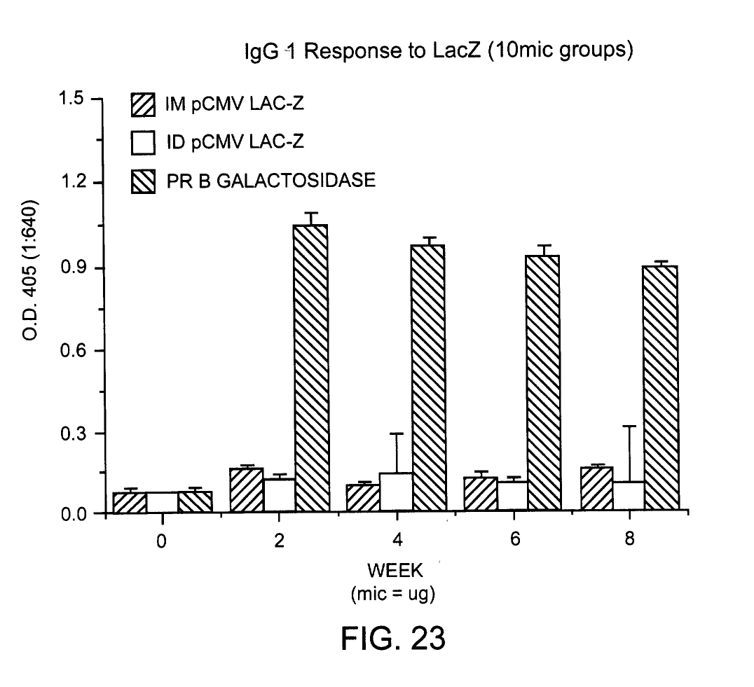

As shown in FIG. 22, only the mice that received the plasmid by ID injection produced high titers of IgG 2A antibodies. As shown in FIG. 23, immunization of the mice with the enzyme itself (“PR”) induced production of relatively high titers of IgG 1 antibodies. In the IM injected mice, low titers of both IgG 2A and IgG 1 antibodies were produced without apparent selectivity. The data shown in the FIGURE comprise averages of the values obtained from each group of 4 mice.

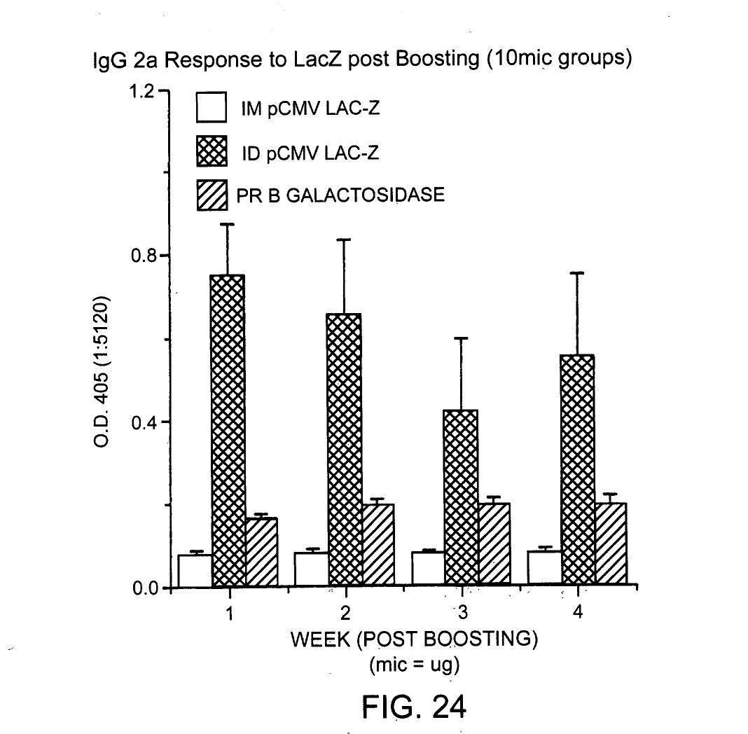

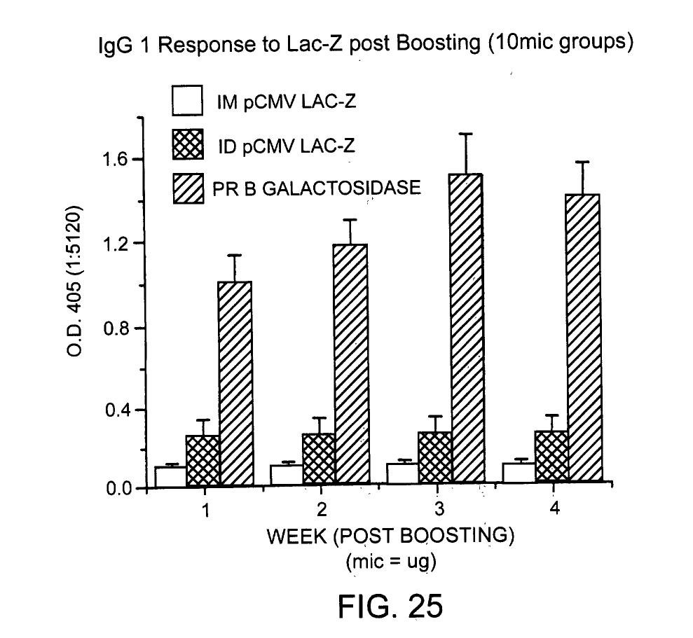

To determine the stability of the antibody response over time, the same group of animals were boosted with 0.5 μg of enzyme injected intradermally. As shown in FIGS. 24 and 25 boosting of ID injection primed animals with the enzyme induced a nearly 10-fold rise in IgG 2A antibody responses (i.e., the antibody titer rose from 1:640 to 1:5120), but did not stimulate an IgG 1 response. These data indicate that the selective TH1 response induced by ID administration of naked polynucleotides is maintained in the host, despite subsequent exposure to antigen.

### Example XIII

Suppression of IgE Antibody Response to Antigen by Immunization with Antigen-Encoding Polynucleotides

Using the experimental protocol described in Examples XI and XII, five to eight week old Balb/c mice were immunized with one of two naked gene expression vectors of the invention: the pCMV-LacZ plasmid described in Example XI or a control plasmid, pCMV-BL (which does not encode for any insert peptide and does not contain immunostimulatory polynucleotides). A third group of the mice received injections of antigen (β galactosidase). Plasmid DNA was purified and its endotoxin content reduced to 0.5-5 ng/1 mg DNA by extraction with TRITON X-114 (Sigma, St. Louis, Mich.). Before inoculation, pDNA was precipitated in ethanol, washed with 70% ethanol and dissolved in pyrogen-free normal saline.

Immunization was by intradermal injection of plasmid DNA loaded onto separate tynes of a MONOVACC8 multiple tyne device (Connaught Lab, Inc., Swiftwater, Pa.). Briefly, the tyne devices were prepared after extensive washing in DDW and overnight soaking in 0.5% SDS (sulfated dodecyl saline), washed again in DDW, soaked overnight in 0.1N NaOH, washed again in DDW and dried at 37° C. for 8 hours. Six μl of plasmid DNA dissolved in normal saline were pipetted onto the tynes of the tyne device just prior to each inoculation described below. The total amount of pDNA loaded on the device per inoculation was 25 μg each of pCMV-LacZ and pCMV-BL. For purposes of estimating actual doses, it was assumed that less than 10% of the pDNA solution loaded onto the tyne device was actually introduced on injection of the tynes into intradermal tissue.

Each mouse was treated 3 times with 2 inoculations of each plasmid in a one-week interval injected intradermally at the base of the tail. Another group of mice received a single intradermal injection in the base of the tail of 10 μg of β galactosidase protein (dissolved in 50 μl of normal saline) in lieu of pDNA.

Toward inducing an IgE antibody response to subsequent antigen challenge, each group of mice was injected once intraperitoneally with 0.1 ml of phosphate buffered saline (PBS) solution containing 1 μg of antigen (β galactosidase; Calbiochem, San Diego, Calif.) and 3 mg of ALUM aluminum hydroxide as adjuvant (Pierce Chemical, Rockford, Ill.) 14 weeks after the initial immunization. Total IgE was assayed in sera from the mice 4 times over the subsequent 4 consecutive weeks.

IgE was detected using a solid phase radioimmunoassay (PAST) in a 96 well polyvinyl plate (a radioisotopic modification of the ELISA procedure described in Coligan, “Current Protocols In Imnmunology”, Unit 7.12.4, Vol. 1, Wiley & Sons, 1994), except that purified polyclonal goat antibodies specific for mouse ε chains were used in lieu of antibodies specific for human Fab. To detect anti-LacZ IgE, the plates were coated with β galactosidase (10 μg/ml). The lowest IgE concentration measurable by the assay employed was 0.4 ng of IgE/ml.

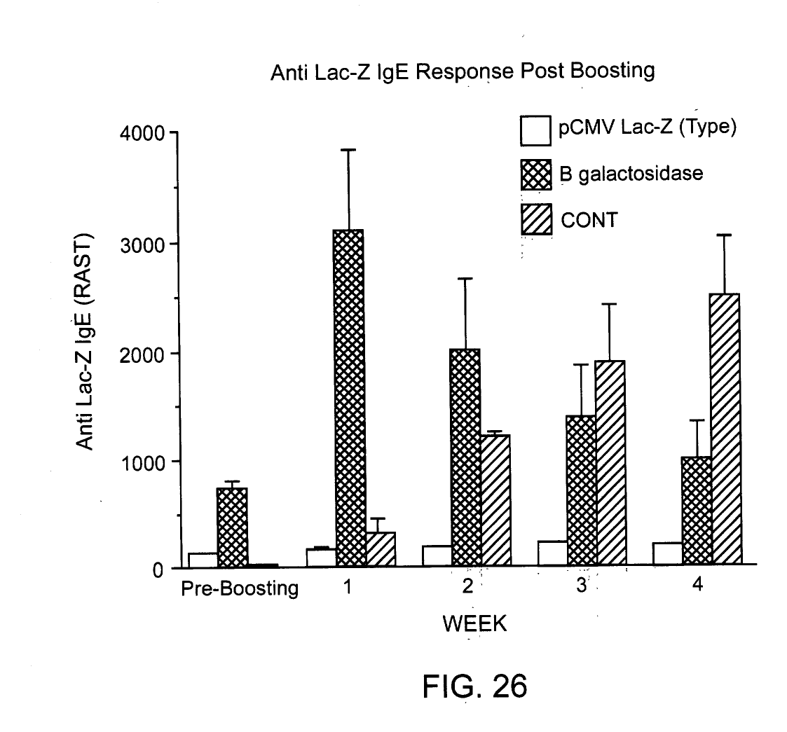

Measuring specifically the anti-antigen response by each group of mice, as shown in FIG. 26, anti-LacZ IgE levels in the plasmid injected mice were consistently low both before and after boosting (averaging about 250 CPM in RAST), while the protein injected mice developed high levels of anti-LacZ, particularly after the first antigen booster injection, when anti-LacZ levels in the mice rose to an average of about 3000 CPM. Consistent with acquisition of tolerance, anti-LacZ IgE levels in the protein injected mice declined over time, but continued to rise in the control mice that had not received any immunization to β galactosidase.

These data show that the plasmid injected mice developed an antigen specific TH1 response to the plasmid expression product, with concomitant suppression of IgE production, while tolerance was acquired in the protein injected mice only after development of substantially higher levels of total and antigen specific IgE antibodies.

### Example XIV

Epidermal Administration of a Naked Gene Expression Vector Using a Chemical Agent to Elicit an Immune Response

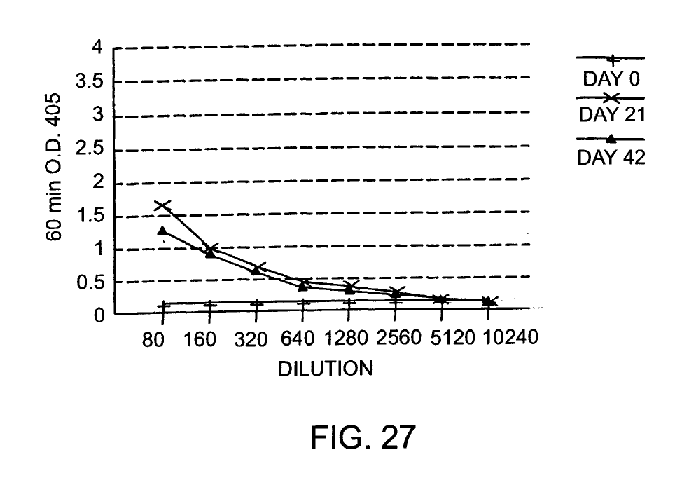

FIG. 27 depicts the results of an ELISA performed as described in Example VII for serum levels of anti-NP IgG following epidermal administration of the pCMV-NP vector described in Example VII in conjunction with the application of a chemical agent.

The plasmid was suspended in 40 μg of an isotonic normal saline solution containing approximately 150 μg of plasmid per milliliter. This solution was absorbed onto the nonadhesive pad of a BAND-AIDS brand bandage (Johnson & Johnson).

A Balb/c mouse was shaved along the base of its tail and a commercially available keratinolytic agent (here, the previously described depilatory cream sold under the trademark NAIR™) was applied to the shaved skin. After several minutes, the keratinolytic agent was washed off of the skin and the plasmid-containing bandage applied thereto. As shown in FIG. 27, the treated animal developed serum anti-NP IgG at a titer of 1:640.

### Example XV

Enhancement of Interferon and Cytokine (IL-4) Production in Animals Immunized with Immunostimulatory Polynucleotide Containing Plasmids

Two groups of mice were immunized with either pCMV-LacZ or pKCB-LacZ as described in Example IX. A third group of mice received a combination dose of pKCBLacZ and pUC-19 as described in Example VII. After sacrifice, splenocytes were removed and challenged in vitro with β-galactosidase antigen. The release of IFN-γ and IL-4 into supernatants from the antigen challenged cells was measured.

Mice immunized with pKCB-LacZ alone produced little IFN-γ and IL-4 as compared to mice immunized with pCMV-LacZ or the combination pKCB-LacZ/pUC-19 dose.

### Example XVI

Duration of Effect of Immunostimulatory Nucleic Acid

The duration of an effect of an immunomodulatory nucleic acid molecule on an immune response was investigated. The results indicate that, in this system, a single dose (e.g., a single intraperitoneal dose) of ISS effectively inhibits Th2 responses and induces Th1 responses, as characterized by IL-5 levels, eosinophilia (lung, BAL, bone marrow, and blood) IgE levels, and airway responsiveness to Mch, and that this effect persists for several weeks, but eventually wanes over time such that by 8 weeks, the ISS effect is no longer present.

Materials and Methods

IgE Assay

IgE anti-ova antibody titers were determined using a modified ELISA as previously described. Tighe et al. (2000) J. Allergy Clin. Immunol. 106:124-134. The modification consisted of first absorbing the serum with protein G to remove IgG anti-ova antibodies that compete with IgE antibodies for antigen in the ELISA test. Raz et al. (1996) Proc. Natl. Acad. Sci. USA 93:5141-5145. Aliquots of individual sera were added to a 50% slurry of protein G Sepharose beads (Pharmacia, Piscataway, N.J.) in borate buffered saline (pH 8.5) at a final 1:10 dilution and rotated overnight at 4° C. ELISA plates were coated with 5 μg/mL ova in carbonate buffer (pH 9.0). Each plate contained a standard titration of a protein-G absorbed serum pool that was assigned an IgE value in units/mL that was equal to the reciprocal of the highest dilution that gave an OD reading double that of the background. IgE values (units per mL) of the test sera were calculated relative to this IgE standard serum.

Statistics

Results from the different duration of ISS treated groups were compared by student's t test using a statistical software package (In Stat, San Diego, Calif.). P-values of <0.05 were considered statistically significant. All results are given as mean±SEM.

Oligonucleotides

Endotoxin-free (<1 ng/mg DNA) phosphorothioate ISS-ODN (5′-TGACTGTGAACGTTCGAGATGA-3′; SEQ ID NO:1) or phosporothioate (M)-ODN (5′-TGACTGTGAAGGTTAGAGATGA-3′; SEQ ID NO:4) (Trilink, San Diego, Calif.), as previously described were used in the in vivo and in vitro experiments described below. Broide et al. (1998) J. Immunol. 161:7054-7062.

Animals

Female BALB/c mice (The Jackson Laboratory, Bar Harbor, Me.) were used when they reached 8-10 wk of age. All animal experimental protocols were approved by the University of California, San Diego animal subjects committee.

Mouse Model of Eosinophilic Pulmonary Inflammation

Pulmonary eosinophilia in mice was induced as previously described in this laboratory. Broide et al. (1998) J. Immunol. 161:7054-7062; and Broide et al. (1999) Int. Arch. Allergy Immunol. 118:453-456. In brief, BALB/c mice were sensitized by s.c. injection of 25 μg ovalbumin/1 mg alhydrogel (Aldrich Chemical Co., Milwaukee, Wis.) in 0.9% sterile saline on days 0, 7, 14, and 25. Nonsensitized mice receive 1 mg of alhydrogel in 0.9% saline. On day 26 and day 30, mice (n=4 mice/group) were exposed three times for 30 minutes (at 30 minute intervals) to an aerosol of ovalbumin (ova; 10 mg/ml) in 0.9% saline (non-sensitized control mice received saline only.

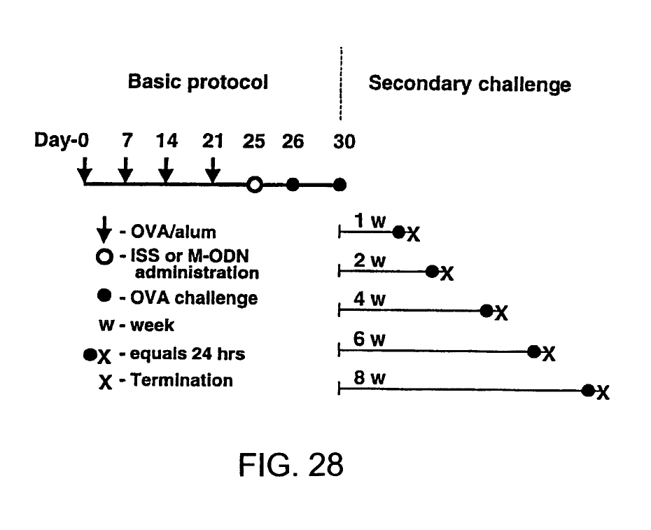

Groups of mice which had been challenged with ova by inhalation on day 26 and day 30 (first series of ova inhalation challenges) were then rechallenged with ova (second series of ova inhalation challenges) at time points 1-8 weeks after the first series of ova inhalation challenges. Twenty four hours after the first or second series of ova inhalation challenges, mice were killed by CO₂ asphyxiation (see FIG. 28 for ova and ISS protocol).

Administration of ISS-ODN

ISS-ODN or M-ODN were injected i.p. (100 μg in 100 μl of sterile, endotoxin-free PBS) once 1 day before the first ova inhalation challenge on day 26. We have previously demonstrated that this dose and timing of ISS administration inhibits subsequent ova aerosol induced eosinophilic inflammation. Broide et al. (1998) J. Immunol. 161:7054-7062; and Broide et al. (1999) Int. Arch. Allergy Immunol. 118:453-456.

Bronchoalveolar Lavage Eosinophils

Bronchoalveolar lavage (BAL) cells from mice were recovered by lavage with 1 ml of PBS via a tracheal catheter. The resulting BAL cells were immediately separated from BAL fluid by centrifugation (700×g for 5 minutes). An appropriate phosphate-buffered saline dilution of the recovered BAL cells was added to trypan blue, and the viability and total number of BAL white blood cells counted with a hemocytometer. Differential leukocyte counts were performed after brief acetone fixation and staining of the BAL cells with May-Grünwald-Giemsa stains. The percentage ofeosinophils, neutrophils and mononuclear cells present on each slide were assessed by counting a minimum of 300 cells in random high power fields using a light microscope (40×magnification).

Lung Tissue Eosinophils

Lung tissues embedded in OCT in 10×50×50-mm tissue wells were cryosectioned at 10 μm and acetone fixed onto poly(L-lysine)-coated slides. Total eosinophil numbers were enumerated by detection of eosinophil peroxidase using DAB staining and microscopic examination, as described in this laboratory. Broide et al. (1998) J. Immunol. 161:7054-7062. Slides were incubated at room temperature for 1 min in the presence of cyanide buffer (10 mM potassium cyanide, pH 6), rinsed in PBS, and incubated for 10 min with the peroxidase substrate DAB (Vector Lab, Burlingame, Calif.). Slides were subsequently washed in PBS, counter-stained with hematoxylin, air dried, and examined by light microscopy (×40 magnification). Five random fields were selected and eosinophils were counted (cells staining brown) to determine total eosinophil number per microscope field.

Peripheral Blood Eosinophils

Blood was collected from the carotid artery. RBC were lysed using a 1:10 solution of 100 mM potassium carbonate, 1.5 M ammonium chloride. The remaining cells were cytospun (3 min at 500 rpm) onto microscope slides and air dried. Eosinophil counts were performed as described above.

Bone Marrow Eosinophils

Bone marrow cells were flushed from femurs with 1 ml PBS and cytospun onto microscope slides, and separate slides were stained with Wright-Giemsa and DAB for cell differential counts.

Determination of Airway Responsiveness to Methacholine

Airway responsiveness was assessed on day 31 twenty four hours after completion of the first OVA inhalation challenges, or 24 hours after the final ova inhalation challenge 1-8 weeks later using a single chamber whole body plethysmograph obtained from Buxco (Troy, N.Y.), as previously described. Broide et al. (1998) J. Immunol. 161:7054-7062. In this system, an unrestrained, spontaneously breathing mouse is placed into the main chamber of the plethysmograph, and pressure differences between this chamber and a reference chamber are recorded. In the plethysmograph, mice were exposed for 3 minutes to nebulized PBS and subsequently to increasing concentrations of nebulized methacholine (MCh) (Sigma, St. Louis, Mo.) in PBS using an Aerosonic ultrasonic nebulizer (DeVilbiss). Methacholine challenge is carried out to assess the bronchial hyperresponsiveness of a subject. MCh causes the airways to narrow or constrict. After each nebulization, recordings were taken for 3 minutes. Penh (enhanced pause) is an empirical index of the spontaneous breathing pattern. The Penh values measured during each 3-minute sequence are expressed for each MCh concentration (3-24 mg/ml). Broide et al. (1998) J. Immunol. 161:7054-7062. The PC200 concentration of methacholine is the concentration of MCh that causes a 200% increase in Penh from baseline Penh measurements.

Cytokine Assays

Stimulation of splenocytes (5×10⁶/ml) in vitro by ova (100 ng/ml) was performed as described. Broide et al. (1998) J. Immunol. 161:7054-7062. Supernatants (72 h post stimulation) were assayed in duplicate to determine the level of each cytokine (IL-5, interferon-γ) utilizing an ELISA (Pharmigen, San Diego).

Results

Effect of ISS on Th1 vs. Th2 Cytokine Production

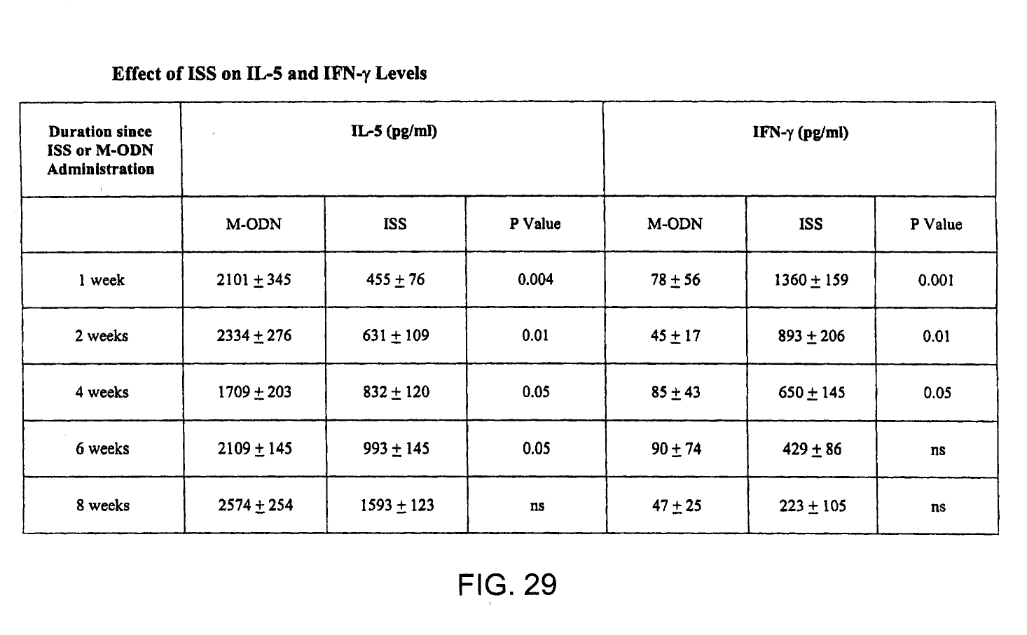

As previously demonstrated a single dose of ISS administered 6 days prior to ova inhalation challenge significantly inhibited IL-5 (a Th2 cytokine response) at 1 week (M-ODN 2101±345 pg/ml IL-5, vs ISS 455±76 pg/ml IL-5) (n=4) (p=0.004). The ability of inhibit IL-5 generation decreased with time from 78% inhibition of IL-5 at 1 week (ISS vs M-ODN)(p=0.004), to 73% inhibition at 2 weeks (p=0.01), 51% inhibition at 4 weeks (p=0.05), 52% inhibition at 6 weeks (p=0.05), and 38% inhibition at 8 weeks (p=ns), as shown in FIG. 29. The amount of IL-5 generated by ISS pretreated mice increased progressively from 455 pg/ml at 1 week to 1593 pg/ml at 8 weeks (n=4) (p=0.05) (FIG. 29) indicating the reversible inhibition of the Th2 cytokine response with time.

The ability of ISS to induce a Th1 response also waned with time indicating the reversible nature of the induction of the Th1 response by a single dose of ISS. For example a single dose of ISS administered on day 25 six days prior to ova inhalation challenge (see experimental design FIG. 28) significantly induced interferon-γ (a Th1 cytokine response) at 1 week (ISS 1360±159 μg/ml interferon-γ, vs M-ODN 78±56 μg/ml interferon-γ) (n=4) (p=0.001). The ability of ISS to induce interferon-γ generation decreased with time from 1360 pg/ml at 1 week to 223 pg/ml at 8 weeks (n=4) (p=0.05) (FIG. 29) indicating the reversible induction of the Th1 cytokine response with time.

Effect of ISS on BAL and Lung Eosinophilic Inflammation

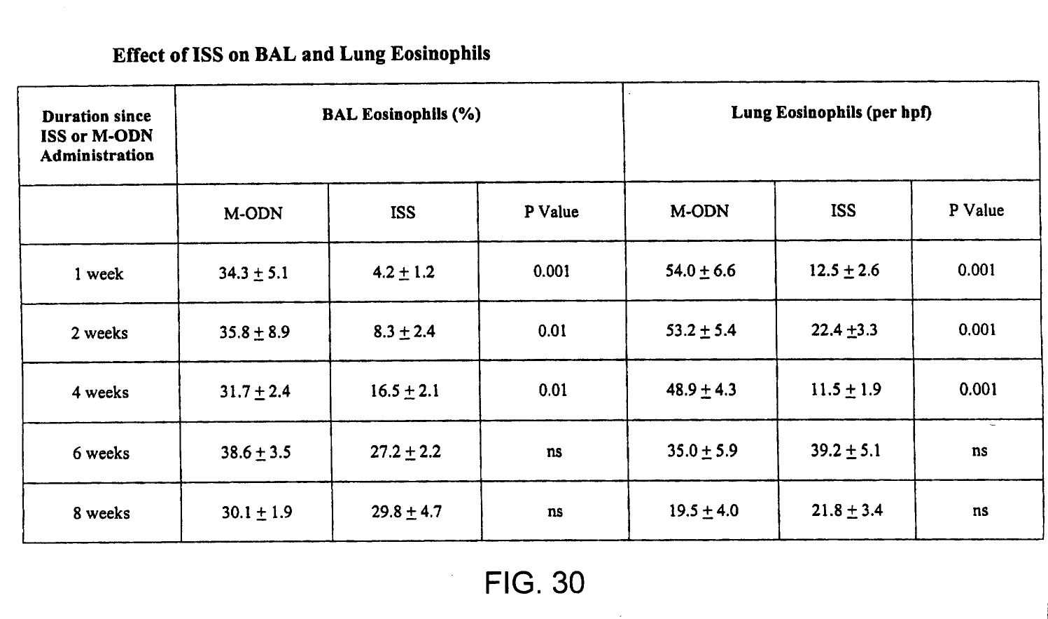

Sensitization and ovalbumin allergen challenge of wild-type mice (n=3 experiments) induced a significant BAL eosinophilia (39.3±5.1% BAL eosinophils) compared to mice that were not sensitized or challenged with ova (1.8±0.5% BAL eosinophils) (p=0.003), or compared to mice immunized with ova and challenged with PBS diluent (5.7±1.4% BAL eosinophils) (p=0.002). Neutrophils comprised less than 2% of BAL cells pre-allergen, post-allergen, or post-diluent challenge. Mononuclear cells comprised the remainder of the BAL cells. As previously demonstrated ISS significantly inhibited pulmonary eosinophilia in ovalbumin challenged mice by 89% at week 1 (M-ODN 34.3±5.1% BAL eosinophils vs ISS 4.2±1.2% BAL eosinophils) (n=3) (p=0.001)(FIG. 30). The ability of a single dose of ISS to inhibit BAL eosinophilia waned with time such that by 8 weeks there was no significant inhibition of ova induced BAL eosinophilia (FIG. 30). The ability of ISS to inhibit BAL eosinophilia decreased with time from 89% inhibition of BALeosinophilia at 1 week (ISS vs M-ODN)(n=3)(p=0.001), to 77% inhibition at 2 weeks (n=3) (p=0.01), 48% inhibition at 4 weeks (n=3) (p=0.01), 29% inhibition at 6 weeks (n=3)(p=ns) and no inhibition at 8 weeks (FIG. 30). ISS reversibly inhibits both the % of eosinophils in BAL as well as the absolute BAL eosinophil count. In the acute ova challenge model BAL eosinophil counts decreased from 0.82±0.14×10⁵ BAL eosinophils in mice treated with M-ODN, to 0.07±0.05×10⁵ BAL eosinophils in mice treated with ISS (p=0.001). At 8 weeks following ISS treatment there was no significant difference in levels of BAL eosinophils in mice treated with M-ODN (0.57±0.15×10⁵ BAL eosinophils) compared to levels in mice treated with ISS (0.68±0.14×10⁵ BAL eosinophils) (p=ns).

A similar response was noted in the effect of ISS on the accumulation of lung eosinophils (FIG. 30). As previously demonstrated ISS significantly inhibited pulmonary eosinophilia in ova challenged mice by 77% at week 1 (M-ODN 54.0±6.6 lung eosinophils/hpf, vs ISS 12.5±2.6 lung eosinophils/hpf) (n=3) (p=0.001) (FIG. 30). The ability of a single dose of ISS to inhibit lung eosinophilia waned with time such that by 8 weeks there was no significant inhibition of ova induced BALeosinophilia. ISS inhibited lung eosinophilia by 77% at 1 week (ISS vs M-ODN)(n=3)(p=0.001), and this inhibition was maintained at 2 weeks (n=3)(p=0.001), and at 4 weeks (n=3)(p=0.001). However, there was no statistically significant inhibition by ISS of lung eosinophilia at 6 weeks (n=3)(p=ns) or at 8 weeks (n=3)(p=ns)(FIG. 30).

Effect of ISS on Bone Marrow and Blood Eosinophils

The number of bone marrow eosinophils were decreased significantly by ISS by 46% at week 1 (ISS vs M-ODN)(n=3)(p=0.01), but not at week 8 (ISS vs M-ODN)(n=3)(p=ns). Similarly the number of peripheral blood eosinophils were decreased significantly by ISS by 63% at week 1 (ISS vs M-ODN)(n=3)(p=0.01), but not at week 8 (ISS vs M-ODN)(n=3)(p=ns).

Effect of ISS on Airway Hyperreactivity to Methacholine

Airway responsiveness to MCh was significantly increased in mice following ova sensitization and ova challenge. Mice sensitized to ova without inhalation challenge, or mice ova challenged without sensitization showed minimal change in Penh in response to MCh (data not shown). As previously reported ISS significantly inhibited airway responsiveness to MCh in ova sensitized and ova challenged mice at 1 week (ISS ova/ova mice MCh 24 mg/ml Penh 1.28±0.06 vs M-ODN ova/ova mice MCh 24 mg/ml Penh 2.94±0.59)(p=0.001). There was no significant difference in baseline Penh pre MCh challenge in ISS vs M-ODN treated mice at week 1 (ISS ova/ova mice baseline Penh 0.34±0.04 vs M-ODN ova/ova mice baseline Penh 0.30±0.01)(p=ns).

In contrast to the inhibition of MCh responsiveness by ISS at week 1, there was no significant difference at 8 weeks post ISS administration in MCh airway responsiveness in ISS treated ova/ova mice as compared to M-ODN treated ova/ova mice (ISS ova/ova mice MCh 24 mg/ml Penh 2.56±0.38 vs M-ODN ova/ova mice MCh 24 mg/ml Penh 2.84±0.68)(p=ns). There was no significant difference in baseline Penh pre MCh challenge in ISS vs M-ODN treated mice at week 8 (ISS ova/ova mice baseline Penh 0.34±0.01 vs M-ODN ova/ova mice baseline Penh 0.33±0.01)(p=ns).

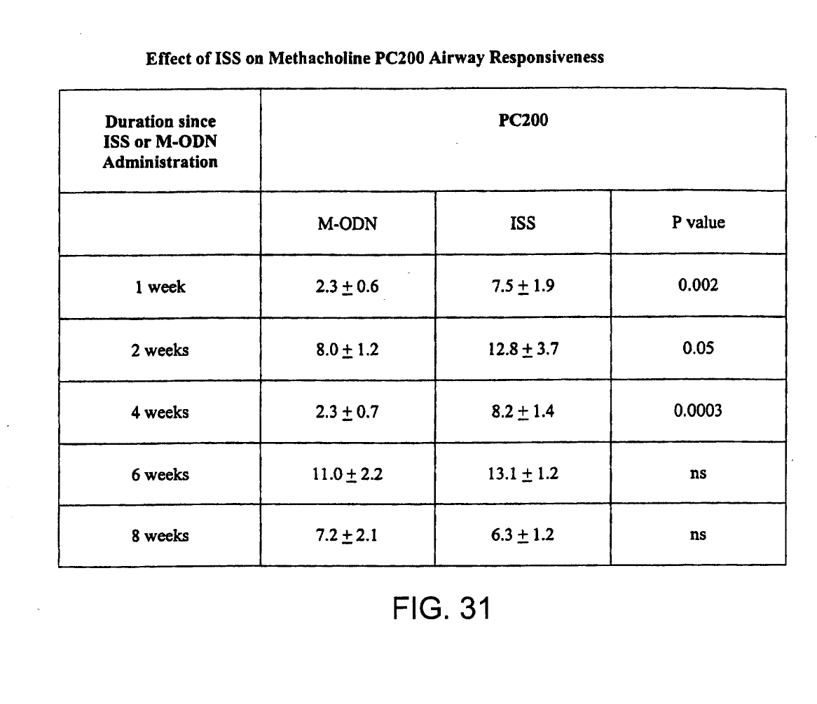

The MCh PC200 (the concentration of MCh that provokes a 200% increase in Penh) was significantly greater in ISS compared to M-ODN treated mice at 1 week (ISS 7.5±1.9 mg/ml MCh, vs M-ODN 2.3±0.6 mg/ml MCh) (p=0.002)(FIG. 31). ISS maintained an increase in the MCh PC200 at 2 weeks (ISS 12.8±3.7 mg/ml MCh, vs M-ODN 8.0±1.2 mg/ml MCh) (p=0.05)(FIG. 31), and 4 weeks (ISS 8.2±1.4 mg/ml MCh, vs M-ODN 2.3±0.7 mg/ml MCh) (p=0.0003)(Table III). At 6 weeks the ISS increase in PC200 was not statistically significantly different from M-ODN (ISS 13.1±1.2 mg/ml MCh, vs M-ODN 11.0±2.2 mg/ml MCh) (p=ns)(FIG. 31), and by 8 weeks ISS had no effect on increasing the PC200 (ISS 6.3±1.2 mg/ml MCh, vs M-ODN 7.2±2.1 mg/ml MCh) (p=ns)(FIG. 31) demonstrating that ISS no longer inhibited MCh airway responsiveness as it did at week 1.

IBE Levels

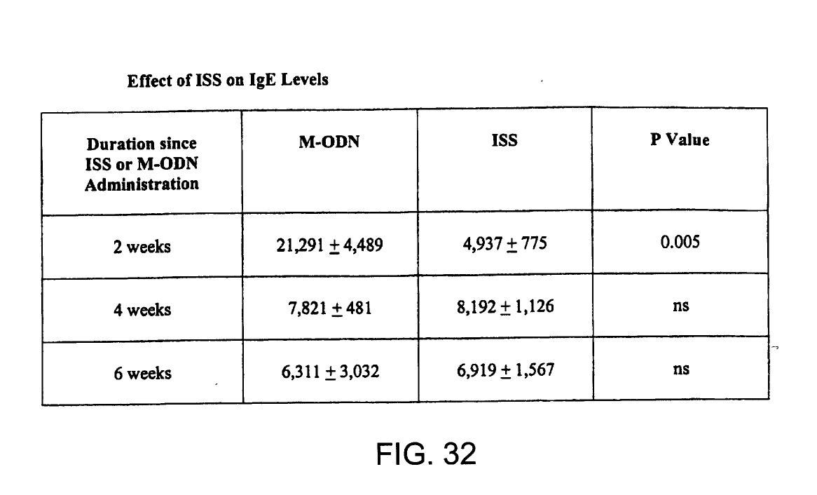

ISS significantly inhibited IgE levels 2 weeks post antigen challenge (ISS IgE level 4,937±775 units/ml vs M-ODN IgE level 20,291±4,489 units/ml)(p=0.005)(FIG. 32). However, there was no inhibition of IgE levels by ISS at 4 weeks (ISS IgE level 8,192±1,126 units/ml vs M-ODN IgE level 7,821±481 units/ml)(p=ns) or at 6 weeks (ISS IgE level 6,919±1,567 units/ml vs M-ODN IgE level 6,311±3,032 units/ml)(p=ns).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of suppressing a symptom of an allergic response in a subject, the method comprising:
administering to an antigen-sensitized mammalian host a first dose of a composition comprising an immunomodulatory nucleic acid; and
administering to the host a second dose of a composition comprising an immumodulatory nucleic acid, wherein the immunomodulatory nucleic acid comprises a nucleotide sequence comprising 5′-CG-3′, and wherein the second dose is administered from about 1 day to about 8 weeks after the first dose.

2. The method of claim 1, wherein the second dose is administered from about 1 day to about 7 days after the first dose.

3. The method of claim 1, wherein the second dose is administered from about 1 week to about 2 weeks after the first dose.

4. The method of claim 1, wherein the second dose is administered from about 2 weeks to about 4 weeks after the first dose.

5. The method of claim 1, wherein the first dose is co-administered with the antigen to which the animal is sensitized.

6. The method of claim 1, wherein the second dose is co-administered with the antigen to which the animal is sensitized.

7. The method of claim 1, wherein the first dose and second dose are co-administered with the antigen to which the animal is sensitized.

8. The method of claim 1, wherein the mammalian host is a human.

9. The method of claim 1, wherein the first and the second doses are administered by inhalation.

10. A method for maintaining suppression of a Th2 immune response in a subject, the method comprising:
administering to a mammalian host a first dose of a composition comprising an immunomodulatory nucleic acid; and
administering to the host a second dose of a composition comprising an immumodulatory nucleic acid, wherein the immunomodulatory nucleic acid comprises a nucleotide sequence comprising 5′-CG-3′, and wherein the second dose is administered from about 1 day to about 8 weeks after the first dose.

11. The method of claim 10, wherein the second dose is administered from about 1 day to about 7 days after the first dose.

12. The method of claim 10, wherein the second dose is administered from about 1 week to about 2 weeks after the first dose.

13. The method of claim 10, wherein the second dose is administered from about 2 weeks to about 4 weeks after the first dose.

14. The method of claim 10, wherein the mammalian host is a human.

15. The method of claim 10, wherein the first and the second doses are administered by inhalation.

16. A method for maintaining stimulation of a Th1 immune response in a subject, the method comprising:
administering to a mammalian host a first dose of a composition comprising an immunomodulatory nucleic acid; and
administering to the host a second dose of a composition comprising an immumodulatory nucleic acid, wherein the immunomodulatory nucleic acid comprises a nucleotide sequence comprising 5′-CG-3′, and wherein the second dose is administered from about 1 day to about 8 weeks after the first dose.

17. The method of claim 16, wherein the second dose is administered from about 1 day to about 7 days after the first dose.

18. The method of claim 16, wherein the second dose is administered from about 1 week to about 2 weeks after the first dose.

19. The method of claim 16, wherein the second dose is administered from about 2 weeks to about 4 weeks after the first dose.

20. The method of claim 16, wherein the mammalian host is a human.
